(19) United States
(12) Patent Application Publication
(10) Pub. No.: US 2025/0073288 A1
(43) Pub. Date: Mar. 6, 2025

(54) Compositions and Methods for Modulating the Intestinal Microbiome

(71) Applicant: Gelesis, LLC, Boston, MA (US)

(72) Inventors: Bryan JONES, Wilmington, MA (US); Alessandro SANNINO, Lecce (IT); Yishai ZOHAR, Brookline, MA (US); Christian DEMITRI, San Pietro In Lama (IT); Elaine CHIQUETTE, Boston, MA (US); Maria RESCIGNO, Milan (IT); Alessandra SILVESTRI, Milano MI (IT); Antonio Gil-Gómez, Seville (ES)

(21) Appl. No.: 18/659,478

(22) Filed: May 9, 2024

Related U.S. Application Data
(63) Continuation of application No. PCT/US22/49797, filed on Nov. 14, 2022.
(60) Provisional application No. 63/335,356, filed on Apr. 27, 2022.
(60) Provisional application No. 63/278,559, filed on Nov. 12, 2021.

(51) Int. Cl. A61K 35/747; A61K 35/745; A61K 47/38; A61P 3/04

(52) U.S. Cl. CPC A61K 35/747; A61K 35/745; A61K 47/38; A61P 3/04

(57) ABSTRACT

The present invention provides compositions and methods for modulating the amount of Akkermansia muciniphila in the gut microbiome of a subject. In a principal embodiment, the invention provides a method of increasing the amount of A. muciniphila is the gut microbiome of a subject, comprising administering to the gastrointestinal tract of the subject an effective amount of a polymer hydrogel having an elastic modulus (G′), as defined herein, of at least about 200 Pa.

### RELATED APPLICATIONS

This application is a continuation of International Application No. PCT/US22/49797, which designated the United States and was filed on Nov. 14, 2022, published in English, which claims the benefit of U.S. Provisional Application No. 63/278,559, filed on Nov. 12, 2021, and U.S. Provisional Application No. 63/335,356, filed on Apr. 27, 2022. The entire teachings of the above applications are incorporated herein by reference.

### BACKGROUND OF THE INVENTION

Akkermansia muciniphila (AM or A. muciniphila), a member of the Verricumicrobia, is an important member of the gut microbiome and is important for the maintenance of gut homeostasis. This bacterial species resides in the mucous lining of the colon, where it produces short chain fatty acids using mucin as an energy source (Collado et al., Appl Environ Microbiol. 2007; 73:7767-70). AM's residence in the gut mucosa places it in close proximity to the intestinal epithelium, and the presence of this species has been shown to benefit intestinal barrier function, which prevents the translocation of pro-inflammatory pathogen-associated molecular patterns (PAMPs) beyond the gut (Ashrafian et al., Gastroenterol Hepatol Bed Bench. 2019; 12:163-8).

Due to its unique protective role in the gut, therapeutic modulation of intestinal A. muciniphila is being studied in several diseases that are related to metabolism, inflammation and immunoregulation (see, for example, Ashrafian et al., Gastroenterol Hepatol Bed Bench. 2019; 12:163-8; Zhou et al., Nutr Metab (Lond). 2020; 17:90; Roshanravan, et al., Arch Physiol Biochem. 2021:1-11; Shih et al., Microorganisms. 2020; 8(9); Kim et al., Appl Environ Microbiol. 2020; 86(7); Grander et al., Gut 2018; 67:891-901; Hu et al., Front Microbiol. 2020; 11:586476; Hanninen et al., Gut 2018; 67:1445-53; Ou et al., Nutr Diabetes 2020; 10:12). While some of these studies examined the potential benefit of administration of pasteurized A. muciniphila, most investigated therapeutic administration of live culture or spores with hopes of increasing the abundance of A. muciniphila in the intestines.

There is a need for new compositions and methods for increasing levels of A. muciniphila in the gut microbiome.

### SUMMARY OF THE INVENTION

The present invention relates to the observation that administration of a polymer hydrogel to the gastrointestinal tract results in increased levels of endogenous A. muciniphila in the gut microbiome.

In one embodiment, the present invention provides compositions and methods for increasing the level of A. muciniphila in the gut microbiome of a subject in need thereof. The method comprises administering to the gastrointestinal tract of the subject an effective amount of a polymer hydrogel.

In one embodiment, the method of the invention comprises administering to the gastrointestinal tract of the subject a polymer hydrogel, a probiotic and/or a prebiotic, wherein the polymer hydrogel and the probiotic and/or prebiotic are administered in amounts which together are effective to increase the level of A. muciniphila in the gut microbiome of the subject.

In another embodiment, the invention provides a composition comprising a polymer hydrogel and a probiotic and/or a prebiotic. Preferably the polymer hydrogel and the probiotic and/or the prebiotic are present in the composition in amounts which together are effective to increase the level of A. muciniphila in the gut microbiome of a subject to whom the composition is administered.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a stacked bar chart showing the relative abundance of A. muciniphila in C57B6 mice treated with either low-fat control diet or high fat diet containing either 2% or 4% GelB. The relative abundance of A. muciniphila is represented by the bottom segment of each bar.

FIG. 2 is a bar chart showing the relative abundance (%) of A. muciniphila in the feces of C57B6 mice after 4 weeks treatment in groups receiving a regular chow diet (chow), a high fat high cholesterol diet (HFD), carboxymethylcellulose (CMC), GelB 2% or GelB 4%. The y axis represents the percent relative abundance of A. muciniphila.

FIG. 3 is a bar chart showing the relative abundance (%) of A. muciniphila in the feces of C57B6 mice after 12 weeks treatment in groups receiving a regular chow diet (chow), a high fat high cholesterol diet (HFD), carboxymethylcellulose (CMC), GelB 2% or GelB 4%. The y axis represents the percent relative abundance of A. muciniphila.

FIG. 4 is a bar chart showing the relative abundance (%) of A. muciniphila in the ilea of C57B6 mice after 4 weeks treatment in groups receiving a regular chow diet (chow), a high fat high cholesterol diet (HFD), carboxymethylcellulose (CMC), GelB 2% or GelB 4%. The y axis represents the percent relative abundance of A. muciniphila.

FIG. 5 is a bar chart showing the relative abundance (%) of A. muciniphila in the ilea of C57B6 mice after 12 weeks treatment in groups receiving a regular chow diet (chow), a high fat high cholesterol diet (HFD), AQUALON 7H4FM sodium carboxymethylcellulose (CMC), GelB 2% or GelB 4%. The y axis represents the percent relative abundance of A. muciniphila.

FIG. 6 is a bar chart showing the relative abundance (%) of A. muciniphila in male DIO-NASH (C57B6/6JRj) mice following 36 weeks of GAN 40% fat diet (baseline) and then 6 weeks of treatment in groups receiving a regular chow diet (chow), GAN 40% fat diet (HFD), or GAN 40% fat diet plus GelB 4%. The y axis represents the percent relative abundance of A. muciniphila.

FIG. 7 is a bar chart showing the relative abundance (%) of A. muciniphila in male DIO-NASH (C57B6/6JRj) mice receiving diet reversal after 6 weeks of treatment in groups receiving a regular chow diet (chow), a high fat high cholesterol diet with a fat content of 20% (20% HFD), a high fat high cholesterol diet with a fat content of 30% (30% HFD), a high fat high cholesterol diet with a fat content of 40% (40% HFD), and each of these groups following treatment with GelB 4%. The y axis represents the percent relative abundance of A. muciniphila.

FIG. 8 is (a) a graph of optical density versus time for samples of A. muciniphila and medium only (20 mL or 40 mL), and 40 mL medium plus 0.2 g GelB and (b) a comparison of the optical densities of these groups at 96 hours.

FIG. 9 is (a) a graph of optical density versus time for samples of A. muciniphila and (i) medium only (20 mL and 40 mL), (ii) 40 mL medium plus GelB, (iii) 40 mL medium plus AQUALON 7H4FM sodium carboxymethylcellulose (7H4), (iv) 40 mL medium plus AVANTRX sodium carboxymethylcellulose (AVANTRX) or (v) 40 mL medium plus citric acid (CA) and (b) a graph comparing the optical densities of each group at 60 hours.

FIG. 10 is (a) a graph of optical density versus time in samples of A. muciniphila and 20 mL or 40 mL medium only or 40 mL medium plus 0.2 g GelB in sterile anaerobic broth supplemented with 0.05% mucins and (b) a graph comparing the optical densities of each group at the final reading.

FIG. 11 is (a) a graph of optical density versus time in samples of A. muciniphila and 20 mL or 40 mL medium only or 40 mL medium plus 0.2 g GelB in sterile anaerobic broth supplemented with 0.025% mucins and (b) a graph comparing the optical densities of each group at the final reading.

FIG. 12 is (a) a graph of optical density versus time in samples of A. muciniphila and 20 mL or 40 mL medium only or 40 mL medium plus 0.2 g GelB in sterile anaerobic broth supplemented with 0.01% mucins and (b) a graph comparing the optical densities of each group at the final reading.

FIG. 13 is (a) a graph of optical density versus time in samples of A. muciniphila and 20 mL or 40 mL medium only or 40 mL medium plus 0.2 g GelB in sterile anaerobic broth supplemented with 0% mucins and (b) a graph comparing the optical densities of each group at the final reading.

FIG. 14 illustrates the experimental design for the study described in Example 7.

FIG. 15 is a graph showing weight gain in C57B6/J mice receiving (a) high fat, high cholesterol diet (HFHCC), (b) HFHCC and A. muciniphila (AM), (c) HFHCC and 4% GelB, (d) HFHCC, 4% GelB and AM, or (e) chow and AM.

### DETAILED DESCRIPTION OF THE INVENTION

The present invention provides compositions and methods for increasing A. muciniphila in the gut microbiome of a subject. Such compositions and methods are useful for treating a variety of diseases and disorders, including overweight, obesity, type 2 diabetes and liver diseases, such as non-alcoholic fatty liver disease and non-alcoholic steatohepatitis.

Clinical studies and preclinical models have demonstrated the therapeutic benefits of increased A. muciniphila levels resulting from administration of live or pasteurized A. muciniphila in a variety of diseases and disorders, including overweight, obesity and type 2 diabetes. For example, a high relative abundance of A. muciniphila was shown in a human trial to be associated with a low risk of obesity; the association declined with aging (Zhou et al., Nutr Metab (Lond). 2020, 17:90). A. muciniphila supplementation is supported for the treatment of obesity and associated disorders (Roshanravan, et al., Arch Physiol Biochem. 2021:1-11). In addition, A. muciniphila abundance was negatively correlated with HbA1c in human subjects with Type 2 diabetes, indicating that A. muciniphila improves glucose homeostasis in such patients (Shih C T, et al., Microorganisms. 2020; 8(9)).

Oral administration of A. muciniphila also lowered serum triglycerides and increased SREBP, a regulator of triglyceride synthesis in the liver in mice on a high fat diet (Kim S, et al., Appl Environ Microbiol. 2020; 86(7)).

In one embodiment, the present invention provides a method for increasing the A. muciniphila level in the gut microbiome in a subject in need thereof. The method comprises the steps of administering to the gastrointestinal tract of the subject an effective amount of a polymer hydrogel. In a preferred embodiment, the method comprises the steps of (a) identifying a subject suffering from a condition which is ameliorated by increased A. muciniphila in the gut microbiome; and (b) administering to the subject a therapeutically effective amount of a polymer hydrogel.

In another embodiment, the present invention provides a method for increasing the A. muciniphila level in the gut microbiome in a subject in need thereof. The method comprises the steps of administering to the gastrointestinal tract of the subject (i) a polymer hydrogel and (ii) a probiotic, wherein the polymer hydrogel and probiotic are administered in amounts which are effective to increase the A. muciniphila level in the subject's gut microbiome. In a preferred embodiment, the method comprises the steps of (a) identifying a subject suffering from a condition which is ameliorated by increased A. muciniphila in the gut microbiome; and (b) administering to the subject (i) a polymer hydrogel and (ii) a probiotic and/or a prebiotic, wherein the polymer hydrogel and the probiotic and/or prebiotic are administered in amounts which are therapeutically effective amount of a polymer hydrogel.

In certain embodiments, the subject is in need of an increased gut microbiome A. muciniphila level to treat overweight, obesity, type 2 diabetes, NAFLD and/or NASH.

In a further embodiment, the invention provides a composition comprising a polymer hydrogel as described herein and a probiotic. Preferably, the composition comprises amounts of the polymer hydrogel and the probiotic which together are effective to increase the A. muciniphila level of the gut microbiome of a subject upon administration of the composition to the subject's gastrointestinal tract.

### Definitions

As used herein, the singular forms “a”, “and”, and “the” include plural referents unless the context clearly dictates otherwise. Thus, for example, reference to “a biomarker” includes a plurality of such biomarkers.

An “effective amount”, as used herein, refers to that amount which provides a desired effect for a given condition and administration regimen; for example, an amount sufficient to increase A. muciniphila in the gut microbiome of a subject. In certain embodiments, the effective amount is a “therapeutically effective amount”, that is, an amount which induces an increase in A. muciniphila in the gut microbiome sufficient to inhibit or reduce a disease or disorder or one or more symptoms thereof. Such an amount can be, for example, a quantity of a polymer hydrogel as described herein shown or predicted to produce a desired therapeutic effect in association with any required additive or diluent, i.e., a carrier or administration vehicle. Further, it is intended to mean an amount sufficient to reduce or prevent a clinically significant deficit in the activity, function and response of the subject. Alternatively, a therapeutically effective amount is sufficient to cause an improvement in a clinically significant condition in a subject, either as a single agent or in combination with one or more additional agents for treating the condition. As is appreciated by those skilled in the art, the amount of a compound may vary depending on its specific activity. Suitable dosage amounts may contain a predetermined quantity of active composition calculated to produce the desired therapeutic effect in association with the required diluent.

The term “subject” or “patient” refers to a human, primate, non-human primate, laboratory animal, farm animal, livestock, or a domestic pet. In preferred embodiments, the subject is a human.

The term “treat” or “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “condition which is ameliorated by increased A. muciniphila in the gut microbiome” is a disease or disorder in which an increase in A. muciniphila in the gut microbiome confers a clinical benefit, i.e., reduces the clinical signs and/or symptoms of the condition. Such conditions include overweight, obesity, Type 2 diabetes, hypertriglyceridemia, liver diseases, including NAFLD and NASH, influenza, bone fracture and intestinal infection.

As used herein, a “polymer hydrogel” is a crosslinked hydrophilic polymer or combination of two or more hydrophilic polymers that is capable of retaining a large relative volume of aqueous solution. The polymer can be branched, linear or a mixture of branched and linear polymers, e.g., about 1, 2, 3, 4, 5, 10, 15, 20, 30, 40, 50, 60, 70, 80, 90, 95, 96, 97, 98, 99, or 100% (w/w) linear versus branched. In preferred embodiments, the hydrophilic polymer or polymers are crosslinked, for example, via physical, ionic, or covalent crosslinks. Polymer hydrogels can have various amounts of cross-linking, depending on the desired physical properties of the polymer hydrogel. Preferably polymer hydrogels used in the methods of the invention have elastic properties that are optimized for increasing the amount of A. muciniphila in the gut microbiome in accordance with the invention. The elastic properties of the polymer hydrogels of use in the methods of the invention are related to their macromolecular structure, including the degree of cross linking, type of crosslinking agent, molecular weight, and structure of the backbone. Preferably, the polymer hydrogel does not include a plasticizer.

The term “simulated gastric fluid/water (1:8)” and the equivalent term “SGF/water (1:8)”, as used herein, refer to a solution prepared according to the method described in Example 2.

As used herein, the “media uptake ratio” or “MUR” of a crosslinked polymer is a measure of the ability of a crosslinked polymer to absorb a specified aqueous medium according to the equation:

$$M U R = ( {W}_{swollen} - {W}_{d r y} ) / {W}_{d r y}$$

where W_{dry} is the weight of the initial dry crosslinked polymer sample and W_{swollen} is the weight of the crosslinked polymer at equilibrium swelling. Unless otherwise noted, a reference herein to media uptake ratio or MUR refers to the value obtained in SGF/water (1:8) according to the method described in Example 2. It is to be understood that the units for MUR values reported herein are g/g.

As used herein, the “elastic modulus” or G′ is determined for a crosslinked polymer swollen in SGF/water (1:8) according to the method described in Example 2.

As used herein, the “tapped density” of a sample is determined according to the method described in Example 2.

As used herein, the “water content” or the “loss on drying” of a sample is determined according to the method described in Example 2.

As used herein, the term “hydrophilic polymer” refers to a polymer which is substantially water-soluble and, preferably, includes monomeric units which are hydroxylated. A hydrophilic polymer can be a homopolymer, which includes only one repeating monomeric unit, or a copolymer, comprising two or more different repeating monomeric units. In certain embodiments, the hydrophilic polymer is an addition polymer or a condensation polymer. A portion or all of the repeating units of a hydrophilic polymer comprise a polar functional group, for example, an acidic, basic or neutral hydrophilic functional group, for example, hydroxyl; carboxyl; sulfonate, phosphonate; guanidine; amandine; primary, secondary, or tertiary amino; or quaternary ammonium. In a preferred embodiment, the hydrophilic polymer is hydroxylated, such as polyallyl alcohol, polyvinyl alcohol or a polysaccharide. Examples of suitable polysaccharides include modified celluloses, including substituted celluloses, substituted dextrans, starches and substituted starches, glycosaminoglycans, chitosan and alginates.

As used herein, the term “ionic polymer” refers to a polymer comprising monomeric units having an acidic functional group, such as a carboxyl, sulfate, sulfonate, phosphate or phosphonate group, or a basic functional group, such as an amino, substituted amino or guanidyl group. When in aqueous solution at a suitable pH range, an ionic polymer comprising acidic functional groups will be a polyanion, and such a polymer is referred to herein as an “anionic polymer”. Likewise, in aqueous solution at a suitable pH range, an ionic polymer comprising basic functional groups will be a polycation. Such a polymer is referred to herein as a “cationic polymer”. An ionic polymer can also be a polyampholyte, namely a polymer which has both acidic and basic functional groups on the backbone, in a ratio which can differ depending on the type of backbone and type of functional group. As used herein, the terms ionic polymer, anionic polymer and cationic polymer refer to hydrophilic polymers in which the acidic or basic functional groups are not charged, as well as polymers in which some or all acidic or basic functional groups are charged, in combination with a suitable counterion. Suitable anionic polymers include alginate, dextran sulfate, carboxymethylcellulose, hyaluronic acid, polyglucuronic acid, polymanuronic acid, polygalacturonic acid, polyarabinic acid; chrondroitin sulfate and dextran phosphate. Suitable cationic polymers include chitosan and dimethylaminodextran. A preferred ionic polymer is carboxymethylcellulose, which can be used in the acid form, or as a salt with a suitable cation, such as sodium or potassium.

The term “nonionic polymer”, as used herein, refers to a hydrophilic polymer which does not comprise monomeric units having ionizable functional groups, such as acidic or basic groups. Such a polymer will be uncharged in aqueous solution. Examples of suitable nonionic polymers for use in the present method are polyallylalcohol, polyvinylalcohol, starches and substituted starches, such as corn starch and hydroxypropylstarch, mannans, glucomannan, acemannans, alkylcelluloses, such as C₁-C₆-alkylcelluloses, including methylcellulose, ethylcellulose and n-propylcellulose; substituted alkylcelluloses, including hydroxy-C₁-C₆-alkylcelluloses and hydroxy-C₁-C₆-alkyl-C₁-C₆-alkylcelluloses, such as hydroxyethylcellulose (HEC), hydroxy-n-propylcellulose, hydroxy-n-butylcellulose, hydroxypropylmethylcellulose, and ethylhydroxyethylcellulose.

### Polymer Hydrogels

In certain embodiments, the polymer hydrogel of use in the methods and compositions of the invention comprises a crosslinked hydrophilic polymer. The polymer hydrogel can be, for example, a crosslinked polyacrylate, a crosslinked polymethacrylate or a crosslinked copolymer of either arcrylate or methacrylate with a neutral monomer, such as acrylamide or methacrylamide. Such polymers and copolymers can be crosslinked using methods known in the art. In certain embodiments, the polymer hydrogel comprises polyethylene glycol diacrylate (PEGDA). Preferably, the average molecular weight of PEGDA ranges from about 250 Da to about 20,000 Da. Preferably the average molecular weight of PEGDA is 250 DA, 575 Da, 700 Da, 750 Da, 1000, Da, 2000 Da, 6,000 Da, 10,000 Da or 20,000 Da.

In preferred embodiments, the polymer hydrogel is biocompatible, that is, the polymer hydrogel is substantially nontoxic when in contact with cells and bodily tissues. More preferably, the polymer hydrogel is formed of materials which are known to be substantially nontoxic when administered to the gastrointestinal tract, such as materials which are generally regarded as safe (GRAS) by the US Food and Drug Administration or materials which are used in food products.

In certain embodiments the polymer hydrogel is a crosslinked polypeptide or a crosslinked combination of two or more polypeptides. The polypeptide can be, for example, a synthetic polypeptide, a protein, such as a globular protein, or a combination of one or more synthetic peptides and one or more proteins. In certain embodiments, the polymer hydrogel is a composite formed by crosslinking a polypeptide and a polysaccharide or synthetic polymer. Suitable examples of crosslinked polypeptides are described in Schloss et al., Adv. Exp. Med. Biol. 2016, 940:167-177; Wu et al., Nature Communications 2018, 9: 620; Jonker et al., Chem. Mater. 2012, 24:759-773; Katyal, et al., Current Opinion in Structural Biology 2020, 63:97-105; U.S. Pat. No. 10,925,999; US Published Application No. 2021/0154364 and U.S. Pat. No. 8,378,022, the contents of each of which are incorporated by reference herein in their entirety.

In certain embodiments, the polymer hydrogel comprises a crosslinked polysaccharide. Polysaccharides which can be used in the polymer hydrogels of the invention include modified celluloses, such as cellulose esters and ethers. Cellulose esters include cellulose acetate, cellulose acetate propionate and cellulose acetate butyrate. Cellulose ethers include alkylcelluloses, such as C₁-C₆-alkylcelluloses, including methylcellulose, ethylcellulose and n-propylcellulose; substituted alkylcelluloses, including hydroxy-C₁-C₆-alkylcelluloses and hydroxy-C₁-C₆-alkyl-C₁-C₆-alkylcelluloses, such as hydroxyethylcellulose, hydroxy-n-propylcellulose, hydroxy-n-butylcellulose, hydroxypropylmethylcellulose, ethylhydroxyethylcellulose and carboxymethylcellulose; starches and substituted starches, such as corn starch, hydroxypropylstarch and carboxymethylstarch; substituted dextrans, such as dextran sulfate, dextran phosphate and diethylaminodextran; glycosaminoglycans, including heparin, hyaluronan, chondroitin, chondroitin sulfate and heparan sulfate; and polyuronic acids, such as polyglucuronic acid, polymanuronic acid, polygalacturonic acid and polyarabinic acid.

The polymer hydrogels of the invention can be covalently cross-linked or non-covalently cross-linked. Preferably, the polymer hydrogel is covalently crosslinked. Covalent crosslinking can be achieved using a bifunctional cross-linking agent (also referred to herein as a bifunctional “cross-linker”) or a multifunctional crosslinking agent, or by direct reaction of functional groups on two different polymer strands. Typical covalent cross-linkers of the present invention include, for example, homobifunctional cross-linkers with reactive functional groups, such as diglycidyl ethers, substituted and unsubstituted di-N-hydroxy succinimides (NHS), diisocyanates, diacids, diesters, diacid chlorides, dimaleimides, diacrylates, and the like. Heterobifunctional cross-linkers can also be utilized. Heterobifunctional cross-linkers usually include molecules that contain different reactive functional groups to accomplish the cross-linking, for example, combining NHS and maleimide, an acid and ester, etc. Covalent crosslinking can also be achieved by irradiation of a hydrophilic polymer or a combination of hydrophilic polymers, for example with x-rays or an electron beam.

Polymer hydrogels comprising a non-covalent cross-linked hydrophilic polymer, e.g., crosslinked via ionic bonds, hydrogen bonds, hydrophobic interactions and/or other intramolecular associations are also contemplated for use in the practice of the invention.

Preferred polymer hydrogels of the invention are crosslinked using a crosslinking agent such as a polycarboxylic acid. As used herein, the term “polycarboxylic acid” refers to an organic acid having two or more carboxylic acid functional groups, such as dicarboxylic acids, tricarboxylic acids and tetracarboxylic acids, and also includes the anhydride and acyl chloride forms of such organic acids. Dicarboxylic acids include oxalic acid, malonic acid, maleic acid, fumaric acid, malic acid, succinic acid, glutaric acid, adipic acid, pimelic acid, suberic acid, azelaic acid, sebacic acid, phthalic acid, o-phthalic acid, isophthalic acid, m-phthalic acid, and terephthalic acid. Preferred dicarboxylic acids include C₄-C₁₂-dicarboxylic acids. Suitable tricarboxylic acids include citric acid, isocitric acid, aconitic acid, and propane-1,2,3-tricarboxylic acid. Suitable tetracarboxylic acids include pyromellitic acid, 2,3,3′,4′-biphenyltetracarboxylic acid, 3,3′,4,4′-tetracarboxydiphenylether, 2,3′,3,4′-tetracarboxydiphenylether, 3,3′,4,4′-benzophenonetetracarboxylic acid, 2,3,6,7-tetracarboxynaphthalene, 1,4,5,7-tetracarboxynaphthalene, 1,4,5,6-tetracarboxynaphthalene, 3,3′,4,4′-tetracarboxydiphenylmethane, 2,2-bis(3,4-dicarboxyphenyl)propane, butanetetracarboxylic acid, and cyclopentanetetracarboxylic acid. Preferred polycarboxylic acids include aliphatic C₂-C₁₂ di-, tri- and tetracarboxylic acids. A particularly preferred polycarboxylic acid is citric acid.

Preferably, a polymer hydrogel of the invention is covalently cross-linked. Preferably the polymer hydrogel has an elastic modulus (G′) when swollen in SGF/water (1:8) of at least 200 Pa or at least 500 Pa, as determined according to the method described in Example 2. Preferably, a polymer hydrogel of the invention has a G′ when swollen in SGF/water (1:8) of at least about 500 Pa, preferably at least about 700, preferably at least about 800, preferably at least about 1000 Pa, preferably at least about 1500 Pa, preferably at least about 2000 Pa, preferably at least about 3000 Pa at least about 3500 Pa, preferably at least about 4000 Pa preferably at least about 4500 Pa, preferably at least about 5000 Pa preferably at least about 5500 Pa, preferably at least about 6000 Pa, preferably at least about 6500 Pa, preferably at least about 7000 Pa, preferably at least about 7500 Pa, preferably at least about 8000 Pa, preferably at least about 8500 Pa. Preferably, the polymer hydrogel is crosslinked carboxymethylcellulose having a G′ when swollen in SGF/water (1:8) from about 500 Pa to about 1500 Pa, from about 500 Pa to about 800 Pa, from about 500 Pa to about 1000 Pa, from about 1500 Pa to about 8000 Pa, from about 5000 Pa to about 8000 Pa, from about 5000 Pa to about 5500 Pa, from about 6000 Pa to about 8000 Pa or from about 6500 Pa to about 8000 Pa.

Preferably, a covalently cross-linked polymer hydrogel of the invention has an elastic modulus (G′) when swollen in SGF/water (1:8) of at least about 200 Pa to about 10,000 Pa. In certain embodiments the covalently cross-linked polymer hydrogel of the invention has an elastic modulus (G′) when swollen in SGF/water (1:8) of about 200 Pa to about 5000 Pa, about 500 Pa to about 9,000 Pa, about 800 Pa to about 8,000 Pa, about 1,000 Pa to about 6,000 Pa, about 500 Pa to about 6,000 Pa, from about 500 Pa to about 3,000 Pa, from about 500 Pa to about 2500 Pa, from about 1,000 Pa to about 10,000 Pa, from about 1,000 Pa to about 8,000 Pa, from about 1,000 Pa to about 5500 Pa, from about 1,200 Pa to about 10,000 Pa or from about 1,200 Pa to about 8000 Pa. In certain embodiments, the polymer hydrogel has an elastic modulus (G′) when swollen in SGF/water (1:8) of about 200 Pa to about 1000 Pa, about 200 Pa to about 1200 Pa, about 200 Pa to about 1500 Pa, about 200 Pa to about 1800 Pa, about 200 Pa to about 2000 Pa or about 200 Pa to about 2400 Pa.

Preferred polymer hydrogels have similar elastic and/or absorbency properties when swollen in SGF/water (1:8) and simulated intestinal fluid (SIF). For example, preferred polymer hydrogels have a G′ when swollen in SIF which is within ±20% of the G′ when swollen in SGF/water (1:8). Preferred polymer hydrogels further have an MUR in SIF which is within ±20% of the MUR in SGF/water (1:8).

Preferably the polymer hydrogel of the invention maintains the preferred elastic modulus (G′) properties during transit through the GI tract. Preferably the polymer hydrogel remains stable during transit through the GI tract including the colon. Alternatively, the polymer hydrogel remains intact in the stomach and small intestine and degrades or partially degrades during the transit through the colon. Alternatively, the polymer hydrogel partially degrades during transit through the small intestine and or the colon. Partial degradation of the polymer hydrogel may be achieved by stabilizing copolymers in the network, where one or more of the polymers are degradable in different parts of the GI tract. An example of such a mechanism, without limitation, is the crosslinking of CMC and chitosan, or CMC and glucomannan, for example, with citric acid or a bifunctional polyethylene glycol (PEG). These copolymer backbones provide such a partial degradation approach. The CMC portion will degrade in the colon while the chitosan or glucomannan portion will remain stable, maintaining a high elastic modulus. Alternatively, partial degradation can be achieved by homopolymers, using different cross-linkers, when one or more of the cross linkers are degradable in different GI tracts. An example is a cellulose derivative crosslinked with citric acid and bifunctional PEG, where the citric acid crosslinks will degrade while the PEG crosslinks will not. Partial degradation may be achieved by a combination of the techniques described above. Once the polymer hydrogel is partially degraded, either by polymer and/or cross linker degradation, the elastic response to deformation, which is entropic in nature, decreases. Thus, the elastic modulus decreases accordingly. Partial degradation can be used as a tool to adjust the elastic modulus of the polymer hydrogels described in these methods during their transit in different GI tracts. In addition to the ionic polymers discussed below suitable polymers of the invention include the following polymers in crosslinked or uncrosslinked form and include uncrosslinked polymers capable of self-crosslinking once deployed in the GI tract form including but are not limited to: HEC, chitosan, glucomannan, starch, acrylates microcrystalline cellulose, psyllium, and guar gum.

One preferred crosslinker is poly(ethylene glycol) diglycidyl ether (PEGDE). The term “bifunctional polyethylene glycol” and “bifunctional PEG” are used interchangeably herein and refer to a polyethylene glycol polymer which is functionalized at each end with a terminal reactive functional group. Suitable reactive groups include those which are able to react with complementary groups in the polysaccharide, such as hydroxyl, carboxyl and amino groups, to form a covalent bond. Suitable such groups include azide, thiol, succinimide, epoxide, carboxy, amino, ethenyl, ethynyl, nitrophenyl, and bromoalkyl groups. Preferably, the functional group is stable in water at neutral pH. A preferred functional group is epoxide. The PEG unit of the bifunctional PEG can be of any suitable length and is generally characterized by the number average molecular weight (Mₙ) of the bifunctional PEG. In certain embodiments, the bifunctional PEG has an Mₙ from about 150 Da to about 1,000,000 DA, preferably from 200 Da to 100,000 Da, preferably from 250 Da to 50,000 Da, preferably from 200 Da to 10,000 Da, more preferably from 250 Da to 5000 Da, 400 Da to 2500 Da, 250 Da to 1000 Da, 350 Da to 650 Da, 450 Da to 550 Da or about 500 Da to about 550 Da. Preferably the bifunctional PEG is poly(ethylene glycol) diglycidyl ether (PEGDE) having a molecular weight from about 450 Da to about 600 Da, or about 500 Da to about 550 Da or about 520 Da to about 530 Da. Preferably PEGDE has an average molecular weight from about or about 400 Da to about 10,000 Da, preferably about, 400 Da to about 8,000 Da, preferably about 400 Da to 6,000 Da, preferably about 460 Da to about 4,600 Da, preferably about 460 Da to about 3,000 Da. Preferably, the bifunctional PEG is PEGDE and the weight ratio of the polymer(s), for example, polysaccharide(s) to PEGDE in the solution of step (1) is from about 20 w/w to about 20000 w/w, preferably about 50 w/w to about 10000 w/w and more preferably about 100 w/w to about 1000 w/w.

Preferably, the polymer hydrogel of the invention comprises an ionic polymer, preferably an anionic polymer, and most preferably, carboxymethylcellulose. Preferably, the anionic polymer is carboxymethylcellulose which is covalently crosslinked with citric acid or a bifunctional PEG as described herein.

In certain embodiments, the polymer hydrogel of the invention comprises an ionic polymer and a non-ionic polymer. The ionic polymer is preferably an anionic polymer, and most preferably, carboxymethylcellulose. The non-ionic polymer is preferably a non-ionic polysaccharide, such as a substituted cellulose, glucomannan, guar gum or psyllium. In other embodiments, the non-ionic polymer is a hydroxyalkylcellulose, such as hydroxyethylcellulose (“HEC”) or a hydroxyalkyl alkylcellulose. In certain embodiments, the ionic polymer is crosslinked with the non-ionic polymer, for example, with a crosslinking agent such as a polycarboxylic acid, preferably citric acid, or a bifunctional PEG, such as PEGDE. The weight ratios of the ionic and non-ionic polymers (ionic:non-ionic) can range from about 1:10 to about 10:1, preferably from about 1:5 to about 5:1. In preferred embodiments, the weight ratio is greater than 1:1, for example, from about 2 to about 5. In a particularly preferred embodiment, the ionic polymer is carboxymethycellulose, the non-ionic polymer is hydroxyethylcellulose, and the weight ratio (ionic:nonionic) is about 3:1.

Most preferably, the invention provides a crosslinked carboxymethylcellulose, for example a citric acid crosslinked carboxymethylcellulose, which has an elastic modulus (G′) when swollen in SGF/water (1:8) of at least about 200 Pa, 500 Pa, 1000 Pa or 1500 Pa, as determined according to the method described in Example 2. Preferably, the crosslinked carboxymethylcellulose has a G′ when swollen in SGF/water (1:8) of at least about 200 Pa, at least about 500 Pa, at least about 700, at least about 800, at least about 1000 Pa, at least about 1500 Pa, at least about 2000 Pa, at least about 3000 Pa, at least about 3500 Pa, at least about 4000 Pa, at least about 4500 Pa, at least about 5000 Pa, at least about 5500 Pa, at least about 6000 Pa, at least about 6500 Pa, at least about 7000 Pa, at least about 7500 Pa, at least about 8000 Pa, at least about 8500 Pa. Preferably, the citric acid crosslinked carboxymethylcellulose of the invention has a G′ when swollen in SGF/water (1:8) from about 200 Pa to about 8000 Pa, from about 500 Pa to about 3000 Pa, from about 500 Pa to about 2500 Pa, from about 500 Pa to about 8000 Pa or from about 500 Pa to about 5000 Pa. In certain embodiments, the crosslinked carboxymethylcellulose, for example a citric acid crosslinked carboxymethylcellulose, has an elastic modulus (G′) when swollen in SGF/water (1:8) of about 200 Pa to about 1000 Pa, about 200 Pa to about 1200 Pa, about 200 Pa to about 1500 Pa, about 200 Pa to about 1800 Pa, about 200 Pa to about 2000 Pa or about 200 Pa to about 2400 Pa.

Most preferably, the invention provides a crosslinked carboxymethylcellulose, for example a citric acid crosslinked carboxymethylcellulose having an elastic modulus (G′) when swollen in SGF/water (1:8) of at least about 200 Pa to about 10,000 Pa, preferably at least about 500 Pa to about 9,000 Pa, preferably at least about 800 Pa to about 8,000 Pa, and preferably at least about 1,000 Pa to about 6,000 Pa.

Most preferably, the invention provides a crosslinked carboxymethylcellulose, for example a citric acid crosslinked carboxymethylcellulose having a G′ when swollen in SGF/water (1:8) from about 500 Pa to about 9,000 Pa, from about 500 Pa to about 6,000 Pa, from about 500 Pa to about 5,000 Pa, from about 1,000 Pa to about 10,000 Pa, from about 1,000 Pa to about 8,000 Pa, from about 1,000 Pa to about 5500 Pa, from about 1,200 Pa to about 10,000 Pa or from about 1,200 Pa to about 8000 Pa. Preferred polymer hydrogels have similar elastic and/or absorbency properties when swollen in SGF/water (1:8) and simulated intestinal fluid (SIF). For example, preferred polymer hydrogels have a G′ when swollen in SIF which is within ±20% of the G′ when swollen in SGF/water (1:8). Preferred polymer hydrogels have an MUR in SIF which is within ±20% of the MUR in SGF/water (1:8).

In certain embodiments, the polymer hydrogel is in the form of particles. For example, the hydrogel can be in the form of particles in the size range of 100 μm to 2000 μm or 100 μm to 1000 μm. In certain embodiments, at least 50%, 60%, 70% or 80% of the polymer hydroGel-By mass is in the form of particles in the size range of 100 μm to 2000 μm or 100 μm to 1000 μm. In certain embodiments, at least 85%, 90% or 95% of the polymer hydroGel-By mass is in the form of particles in the size range of 100 μm to 2000 μm or 100 μm to 1000 μm. In certain embodiments, the particles have an average size in the range of about 400 μm to about 800 μm.

In certain embodiments, the polymer hydrogel has a loss on drying of about 30% or less, about 25% or less, about 20% or less, about 15% or less or about 10% or less.

Carboxymethylcellulose is commercially available in a wide range of molecular weights. It is generally most convenient to express the molecular weight of a sodium carboxymethylcellulose in terms of the viscosity of a 1.0% (wt/wt) solution of sodium carboxymethylcellulose in water at 25° C. Carboxymethylcelluloses suitable for use in the present invention preferably form a 1% (wt/wt) solution in water having a viscosity under these conditions from about 50 centipoise (cps) to about 12,000 cps, more preferably from about 500 cps to about 12000 cps. In certain embodiments, the viscosity of the solution under these conditions is from about 1000 cps to about 12000 cps, about 1000 cps to about 2800 cps, about 1500 cps to about 3000 cps, about 2500 to about 6000 cps. In certain embodiments, the viscosity of the solution under these conditions is from about 6000 cps to about 12000 cps. The viscosity of the carboxymethylcellulose solution is determined according to the method set forth in Example 2 which is in accordance with ASTM D1439-03(2008)e1 (ASTM International, West Conshohocken, PA (2008), incorporated herein by reference in its entirety).

In one embodiment, the polymer hydrogel is produced by crosslinking high viscosity carboxymethylcellulose. The high viscosity carboxymethylcellulose can be covalently crosslinked and/or physically crosslinked. For example, the high viscosity carboxymethylcellulose can be covalently crosslinked, for example, with a suitable, preferably physiologically acceptable bifunctional crosslinking agent. In one embodiment, the high viscosity carboxymethylcellulose is crosslinked with a polycarboxylic acid, such as citric acid. In another embodiment, the high viscosity carboxymethylcellulose is crosslinked with a bifunctional PEG, such as PEGDE. Polymer hydrogels formed by crosslinking high viscosity carboxymethylcellulose with citric acid are described in US 2016/0222134, the contents of which are incorporated herein by reference in their entirety.

The term “high viscosity carboxymethylcellulose”, as used herein, refers to carboxymethylcellulose, as the sodium salt, which forms a 1% (wt/wt) solution in water having a viscosity of at least 6000 cps at 25° C. In preferred embodiments, the high viscosity carboxymethylcellulose also has a low polydispersity index, such as a polydispersity index of about 8 or less. Preferably, the high viscosity carboxymethylcellulose preferably forms a 1% (wt/wt) solution in water having a viscosity at 25° C. of at least about 1500, 2000, 3000, 4000, 5000, 6000, 7000, 7500, or 8000 cps. In certain embodiments, the carboxymethylcellulose forms a 1% (wt/wt) aqueous solution having a viscosity of 6000 to about 10000 cps or about 6000 to 12000 cps at 25° C. In certain embodiments, the carboxymethylcellulose forms a 1% (wt/wt) aqueous solution having a viscosity of about 6000 to about 9500 cps or about 7000 to 9500 cps at 25° C. In another embodiment, the carboxymethylcellulose forms a 1% (wt/wt) aqueous solution having a viscosity of about 7000 to about 9200 cps or about 7500 to 9000 cps at 25° C. In yet another embodiment, the carboxymethylcellulose forms a 1% (wt/wt) aqueous solution having a viscosity of about 8000 to about 9300 cps, or about 9000 cps at 25° C. Preferably the carboxymethylcellulose is in the form of the sodium salt. Preferably, the carboxymethylcellulose is sodium carboxymethylcellulose which forms a 1% (wt/wt) aqueous solution having a viscosity of about 7800 cps or higher, for example, from about 7800 to about 12000 cps, or about 8000 cps to about 11000 cps.

In preferred embodiments, the high viscosity carboxymethylcellulose further has a polydispersity index (M_{w}/Mₙ) of about 8 or less, preferably about 7 or less, or 6 or less. In one embodiment, the polydispersity index is from about 3 to about 8, about 3 to about 7, about 3 to about 6.5, about 3.0 to about 6; about 3.5 to about 8, about 3.5 to about 7, about 3.5 to about 6.5, about 3.5 to about 6, about 4 to about 8, about 4 to about 7, about 4 to about 6.5, about 4 to about 6, about 4.5 to about 8, about 4.5 to about 7, about 4.5 to about 6.5, about 4.5 to about 6, about 5 to about 8, about 5 to about 7.5, about 5 to about 7, about 5 to about 6.5, or about 5 to about 6.

Preferably, the crosslinked carboxymethylcellulose, for example a citric acid crosslinked carboxymethylcellulose, when in the form of particles which are at least 95% by mass in the range of 100 μm to 1000 μm with an average size in the range of 400 to 800 μm and a loss on drying of 10% or less (wt/wt), has a G′, media uptake ratio, and tapped density as described below. Such a crosslinked carboxymethylcellulose can be prepared, for example, according to the methods disclosed herein and in US 2016/0354509.
(A) G′: at least about 1500 Pa, 1800 Pa, 2000 Pa, 2200 Pa, 2500 Pa, or 2700 Pa. In certain embodiments, the crosslinked carboxymethylcellulose of the invention has a G′ when swollen in SGF/water (1:8) of at least about 2800 Pa. In certain embodiments, the crosslinked carboxymethylcellulose of the invention has a G′ when swollen in SGF/water (1:8) from about 1800 Pa to about 3000 Pa, about 2000 Pa to about 4000 Pa, from about 2100 Pa to about 3500 Pa, from about 2100 Pa to about 3400 Pa, or from about 2500 Pa to about 3500 Pa.
(B) Media uptake ratio (MUR) in SGF/water (1:8): at least about 40, preferably at least about 50 or 60. In certain embodiments, the crosslinked carboxymethylcellulose has an MUR of about 50 to about 110, about 55 to about 100, about 60 to about 95, about 60 to about 90, or about 60 to about 85.
(C) Tapped density: at least 0.5 g/mL, preferably about 0.55 g/mL to about 0.9 g/mL. In a preferred embodiment, the tapped density is about 0.6 g/mL or greater, for example, from about 0.6 g/mL to about 0.8 g/mL, about 6.5 g/mL to about 7.5 g/mL or about 0.6 g/mL to about 0.7 g/mL.

Preferably, the invention provides a crosslinked carboxymethylcellulose which has a G′ and media uptake ratio as set forth below when in the form of particles which are at least 95% by mass in the range of 100 μm to 1000 μm with an average size in the range of 400 to 800 μm and a loss on drying of 10% or less (wt/wt):
(A) G′ of about 500 Pa to about 8000 Pa and a media uptake ratio of about 40 to 100;
(B) G′ of about 1200 Pa to about 2000 Pa and a media uptake ratio of at least about 75;
(C) G′ of about 1400 Pa to about 2500 Pa and a media uptake ratio of at least about 70;
(D) G′ of about 1600 Pa to about 3000 Pa and a media uptake ratio of at least about 65;
(E) G′ of about 1900 Pa to about 3500 Pa and a media uptake ratio of at least about 60;
(F) G′ of about 2200 Pa to about 4000 Pa and a media uptake ratio of at least 55;
(G) G′ of about 2600 to about 5000 Pa and a media uptake ratio of at least 40;
(H) G′ above 3000 to about 8,000 Pa and a media uptake ratio of at least about 30;
(I) G′ above 4000 to about 10,000 Pa and a media uptake ratio of at least about 20;
(J) G′ above 6000 to about 11,000 Pa and a media uptake ratio of at least about 15;
(K) G′ above 7,000 to about 12,000 Pa and a media uptake ratio of at least about 10.

Preferably, the foregoing citric acid crosslinked carboxymethylcellulose optionally further has a tapped density of at least 0.5 g/mL, preferably about 0.55 g/mL to about 0.9 g/mL. In a preferred embodiment, the tapped density is about 0.6 g/mL or greater, for example, from about 0.6 g/mL to about 0.8 g/mL, about 0.65 g/mL to about 0.75 g/mL or about 0.6 g/mL to about 0.7 g/mL.

Preferably, the crosslinked carboxymethylcellulose has a G′ of at least about 2100 Pa and a media uptake ratio of at least about 75; or a G′ of at least about 2700 Pa and a media uptake ratio of at least about 70.

Unless otherwise noted, all measurements of G′, MUR and tapped density described herein are made on samples of polymer hydrogel, such as crosslinked carboxymethylcellulose, having (1) a loss on drying of 10% (wt/wt) or less; and (2) are in the form of particles which are at least 95% by mass in the size range of 100 μm to 1000 μm with an average size in the range of 400 to 800 μm.

### Probiotics

The probiotic of use in the methods and compositions of the present invention is any probiotic which is useful for increasing the A. muciniphila level of the subject's gut microbiome in combination with the polymer hydrogel. Probiotics for use in the methods and compositions of the invention include those described by Zhou, K., J. Funct Foods 2017, 33:194-201. Suitable examples include viable A. muciniphila, for example isolated and purified A. muciniphila or crude A. muciniphila, for example, in combination with one or more other microorganisms; pasteurized A. muciniphila; and a mixture of Lactobacillus rhamnosus LMG S-29148 and Bifidobacterium animalis subsp. lactis LMG P-281149.

Other suitable probiotics for use in the methods and compositions of the invention include Anaerostipes spp., such as Anaerostipes caccae; Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium spp, such as Eubacterium hallii and Eubacterium rectale; Faecalibacterium spp., such as Faecalibacterium prausnitzii; Fibrobacter succinogenes, Lactobacillus spp., such as Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, and Lactobacillus rhamnosus; Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and any combination of two or more thereof.

The probiotic can be used in the compositions and methods of the invention in the form of active bacteria, live bacteria, heat-inactivated bacteria, pasteurized bacteria, tindallized bacteria, bacterial cell lysate and fractions of the same, bacteria cell-free supernatant, products of bacterial fermentation, bacteria fragments, bacterial microbial metabolites, bacteria-derived outer membrane vesicles (OMVs) and extracellular vesicles, bacteria exopolysaccharides and combinations of two or more thereof. In preferred embodiments, the probiotic is selected from the group consisting of active A. muciniphila, live A. muciniphila, heat-inactivated A. muciniphila, pasteurized A. muciniphila, tindallized A. muciniphila, A. muciniphila cell lysate and fractions of the same, A. muciniphila cell-free supernatant, products of A. muciniphila fermentation, A. muciniphila fragments, A. muciniphila microbial metabolites, A. muciniphila-derived outer membrane vesicles (OMVs), A. muciniphila extracellular vesicles and combinations of two or more thereof.

### Compositions

In one embodiment, the present invention provides a pharmaceutical composition for increasing A. muciniphila in the gut microbiome in a subject. The pharmaceutical composition comprises a polymer hydrogel as described herein in combination with a pharmaceutically acceptable excipient or carrier. The polymer hydrogel present in the pharmaceutical composition can be hydrated or dehydrated, for example, with a water content about 25% or less, preferably 10% or less by weight. Preferably the pharmaceutical composition is suitable for oral administration. For example, the polymer hydrogel can be dehydrated and formulated as capsules, tablets, or sachets. The polymer hydrogel can also be a component of a formulation or device in which it serves as a mucoadhesive. Such devices include patches in which a layer of the polymer hydrogel is affixed to a barrier layer. Upon adhesion of the polymer hydrogel to the intestinal surface, the patch forms a permeability barrier on the portion of the intestinal wall it covers. See, for example, US 2016/0354509, incorporated herein by reference. The polymer hydrogel can be crosslinked in situ or administered in partially crosslinked form. The polymer hydrogel can be administered in dry (xerogel) or partially swollen or swollen form (polymer hydrogel), alone or in combination with foods or beverages, or a combination thereof. For example, the polymer hydrogel can be mixed with the food or as a component of the food, such as food bars, cereals, yogurts with Gel-Bulks, ice creams, and fruit juices, preferably, but not limited to, beverages with acidic pH, such as orange juice or lemon juice. In another embodiment, the polymer hydrogel is provided in a form which allows it to maintain contact with the oral mucosa, for example, chewable formulations and foods such as popsicles.

The pharmaceutical compositions of the invention can further include pharmaceutically acceptable excipients. In certain embodiments, the pharmaceutical composition is suitable for oral administration, for example, in combination with water or an aqueous solution. In other embodiments, the composition is suitable for rectal administration, for example, in the form of a suppository or in a solution for use as an enema.

Preferably, the polymer hydrogel is administered to the gastrointestinal tract of a patient by oral ingestion of a dosage form, such as capsule or tablet. In certain embodiments, the dosage form includes an enteric coating which inhibits release of the polymer hydrogel and, optionally the probiotic, in the stomach but permits release in the small intestine. Typically for an enteric coated capsule, the enteric coating dissolves at the pH of the jejunum (about pH 5.5), ileum (about pH 6) or colon (about pH 6-7). For example, such a dosage can be achieved by coating the polymer hydrogel, and optionally the probiotic, for example in the form of microparticles compressed into a tablet or in a capsule, with a coating that remains intact at the low pH of the stomach, but readily dissolves when the optimum dissolution pH of the coating is reached. The coating may be provided on the capsule directly, allowing capsule dissolution only in the GI region of interest. The coating can be selected such that it dissolves at the pH of the target region of the intestines. Polymer hydrogel release can be also modulated by administering a xerogel formulation which swells only under specific environmental conditions, such as pH, ionic strength, and temperature.

In certain embodiments, the invention provides a pharmaceutical composition or unitary dosage form comprising the polymer hydrogel and the probiotic. Such a pharmaceutical composition can be, for example, any of the pharmaceutical compositions of the polymer hydrogel described above which further comprise the probiotic. Preferably, the composition comprises amounts of the polymer hydrogel and the probiotic which in combination are effective in increasing the A. muciniphila level of the gut microbiome upon administration to the intestinal tract of a subject, such as a human subject.

In one embodiment, the pharmaceutical composition comprises a polymer hydrogel and a probiotic as described herein and is formulated for oral administration, for example as a capsule, such as an enteric capsule. Suitable capsules include, but are not limited to, dry filled (hard gelatin and/or hard HPMC), liquid filled (hard capsules and/or soft-gel capsules), pH sensitive capsules, osmotic-controlled release oral delivery systems, target delivery capsules, polyvinyl acetate capsules (PVA), pullulan capsules, starch capsules, effervescent capsules, fish gelatin capsules, and vegetable gelatin capsules.

The polymer hydrogel and/or probiotic can also be formulated as a powder, in the form of tablets, a gel or a syrup.

In certain embodiments, the polymer hydrogel and/or the probiotic are administered in a food product. See for example, WO 2010/059725, the contents of which are incorporated herein in their entirety. Suitable food products include, but are not limited to, food bars, oatmeal, cereals, dried/candied fruits, candies, yogurt, pudding, milk powders, juices, pastries and sweets, chocolate bars, jams and marmalades, honey, dressings, homogenized foods, and energy drinks.

In certain embodiments, the polymer hydrogel is formulated as a xerogel, for example having a water content less than 25% by weight, preferably less than 20 or 15% by weight and more preferably 10% or less water by weight. In certain embodiments, the polymer hydrogel is administered orally together with an amount of water sufficient to swell the polymer hydrogel in the stomach of the subject.

In certain embodiments, the invention provides a kit comprising a pharmaceutical composition comprising the polymer hydrogel and a pharmaceutical composition comprising the probiotic. The kit preferably further comprises instructions for use in at least one method, such as (1) a method of increasing the A. muciniphila level of the gut microbiome, (2) a method for treating overweight, (3) a method for treating obesity, and/or (4) a method for treating type 2 diabetes.

### Methods for Increasing Gut A. Muciniphila Levels

In certain embodiments, the present invention provides methods for increasing the A. muciniphila level in the gut microbiome of a subject in need thereof. The method comprises administering an effective amount of a polymer hydrogel to the gastrointestinal tract of the subject. In other embodiments, the method comprises administering the polymer hydrogel and at least one probiotic, to the gastrointestinal tract of the subject, wherein the polymer hydrogel and the at least one probiotic are administered in amounts which together are effective to increase the A. muciniphila content of the subject's gut microbiome. The polymer hydrogel and the optional probiotic are administered to the subject's gastrointestinal tract, for example via oral or rectal administration.

In certain embodiments, the invention provides methods for treating a disease or disorder for which increased A. muciniphila in the gut microbiome provides a therapeutic benefit. In one embodiment, the method comprises the step of administering to the gastrointestinal tract of the subject a therapeutically effective amount of a polymer hydrogel as disclosed herein. In another embodiment, the method comprises administering to the gastrointestinal tract of the subject a polymer hydrogel and a probiotic, wherein the polymer hydrogel and the probiotic are administered in amounts which together are therapeutically effective.

In certain embodiments in which the polymer hydrogel is administered in combination with a probiotic, the polymer hydrogel and the probiotic are administered in separate compositions. In this embodiment, the timing of the administration of the polymer hydrogel and the administration of the probiotic is preferably such that both are present together in the subject's intestinal tract for some period of time. For example, the polymer hydrogel and the probiotic can be administered sequentially with a time separation of less than 10 minutes, for example, less than 5 minutes, or with a time separation more than about 10 minutes. For example, the time between the administration of the polymer hydrogel and the probiotic can be more than 10 minutes, more than 15 minutes, more than 30 minutes, more than 45 minutes, more than 1 hour, more than 5 hours, more than 10 hours, more than 1 day, more than 2 days, more than 3 days, or more than 1 week apart. Either the polymer hydrogel or the probiotic can be administered first. For example, the probiotic can be administered to the subject before or after administration of the polymer hydrogel.

When administered as separate compositions, the polymer hydrogel and the probiotic are both administered to the subject's intestinal tract. Each composition can be independently administered orally or rectally. Preferably, both compositions are administered orally.

In certain embodiments, the polymer hydrogel and the probiotic are administered simultaneously, for example, in separate compositions or in a single dosage form, as described above, which is administered orally or rectally. Preferably, the single dosage form is administered orally.

In certain embodiments, the method of the invention further comprises administering to the subject a prebiotic. The prebiotic can be administered as a separate composition or in composition comprising the prebiotic, the polymer hydrogel and, optionally, a probiotic. Prebiotics for use in the methods and compositions of the invention include, but are not limited to, those described by Zhou, K., J. Funct Foods 2017, 33:194-201. Examples of suitable prebiotics include fructooligosaccharides, fermentable oligo-, di- and mono-saccharides and polyols. Other suitable prebiotics include pomegranate extract, resveratrol, polydextrose, yeast fermentate, sodium butyrate, inulin, polyphenol rich extracts, green tea, berberine, rice bran dietary fiber, dietary barley malt melanoidins, flaxseed, fish oil, and cannabidiol. See, for example, Verhoog S, et al., Nutrients. 2019 Jul. 11; 11(7):1565; Anhê F F, et al, Curr Obes Rep. 2015 December; 4(4):389-400; Jeong H W, et al, J Med Food. 2020 August; 23(8):841-851; Dong C, et al, Biomed Pharmacother. 2021 July; 139:111595; Zhang X, et al., J Agric Food Chem. 2019 Nov. 20; 67(46):12796-12805; Aljahdali N, et al., Nutrients. 2020 Jan. 17; 12(1):241; and Yang C. et al., Food Res Int. 2020 May; 131:108994; Silvestri C, et al, Front Pharmacol. 2020 Oct. 8; 11:585096.

### Treatment of Metabolic Disorders

In certain embodiments, the disease or disorder is overweight, obesity, metabolic syndrome and/or type 2 diabetes. In other embodiments, the disease is a liver disease, such as non-alcoholic fatty liver disease (NAFLD) or non-alcoholic steatohepatitis (NASH). In certain embodiments, the subject to be treated has two or more of the foregoing diseases or disorders. In these embodiments, the polymer hydrogel is preferably administered in combination with a probiotic as discussed above. In certain embodiments, the polymer hydrogel and the probiotic are administered in amounts which together are effective to increase the A. muciniphila content of the subject's gut microbiome sufficiently to induce weight loss and/or reduce the signs and symptoms of type II diabetes, metabolic syndrome or liver disease. The subject to be treated can be overweight, with a body mass index (BMI) greater than 25, or obese, with a BMI greater than 30. The subject can be overweight or obese and additionally suffer from prediabetes, metabolic syndrome, or type 2 diabetes. In certain embodiments, the subject is of normal weight, with a BMI of 18 to 25, but suffers from type 2 diabetes. In certain embodiments, the subject is of normal weight, with a BMI of 18 to 25, but suffers from NAFLD or NASH.

In certain embodiments of the method for treating overweight, obesity, type 2 diabetes, and/or NAFLD or NASH, the polymer hydrogel and the probiotic are administered in combination with at least one additional agent which treats overweight, obesity, type 2 diabetes and/or NAFLD or NASH. The polymer hydrogel, the probiotic and the one or more additional pharmacological therapies or active therapeutic drug agents can be administered simultaneously, in either separate or combined formulations, or sequentially at different times separated by minutes, hours or days, but in some way act together to provide the desired therapeutic response.

The polymer hydrogel and the probiotic are preferably administered to the subject following a dosing regimen which effectively increases the amount of A. muciniphila in the gut microbiome of the subject, or for a length of time resulting in a therapeutic benefit in overweight or obesity, such as weight loss, improvement in one or more signs or symptoms of type 2 diabetes, such as improved glycemic control, or improvement in one or more signs or symptoms of NAFLD or NASH. For example, the polymer hydrogel and the probiotic can independently be administered at least once a month, at least once a week, at least once a day, at least twice every day, at least three times every day or more. For example, the subject can be treated for 1 week, 2 weeks, 3 weeks, 4 weeks, 5 weeks, 6 weeks, 7 weeks, 8 weeks, 9 weeks, or 10 weeks or longer. The polymer hydrogel and the probiotic can be administered alone or in combination with other bioactive agents as discussed above.

### Treatment of Cancer

In certain embodiments, the invention provides a method of enhancing the efficacy of immunotherapy in the treatment of cancer in a subject in need thereof. Preferably, the cancer is one which is susceptible to treatment with an immunooncology agent such as an immune checkpoint inhibitor. The method comprises the steps of administering to the subject an effective amount of a polymer hydrogel prior to treatment with an immunooncology agent. Preferably, the polymer hydrogel is administered a sufficient time prior to administration of the immunooncology agent to allow for an increase in the A. muciniphila level of the subject's gut microbiome before administration of the immunooncology agent.

In another embodiment, the invention provides a method of treating a cancer which is susceptible to treatment with an immunooncology agent in a subject in need thereof. The method comprises the steps of (a) administering to the gastrointestinal tract of the subject an effective amount of a polymer hydrogel and (b) administering to the subject a therapeutically effective amount of an immunooncology agent, such as an immune checkpoint inhibitor. The polymer hydrogel is optionally administered in combination with a probiotic and/or a prebiotic as described above.

An “immune checkpoint inhibitor”, as this term is used herein, is an agent that inhibits the activity of an immune checkpoint protein or the associated signaling cascade, and result in restoration of T cell function and an immune response to the cancer cells. Examples of checkpoint proteins include, but are not limited to: CTLA-4, PD-L1, PD-L2, PD-1, B7-H3, B7-H4, BTLA, HVEM, TIM3, GAL9, LAG3, VISTA, KIR, 2B4, CD160, CGEN-15049, CHK 1, CHK2, A2aR, OX40, B-7 family ligands or a combination thereof. Preferably, the immune checkpoint inhibitor interacts with a ligand of a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, OX40, A2aR, B-7 family ligands or a combination thereof. The immune checkpoint inhibitor can be, for example, a biologic therapeutic, such as a protein, a peptide or a nucleic acid, or a small molecule. Preferably, the immune checkpoint inhibitor is a monoclonal antibody, a humanized antibody, a fully human antibody, a fusion protein or a combination thereof.

In certain embodiments, the immune checkpoint inhibitor is an agent which is an inhibitor of PD-1 signaling. For example, the PD-1 signaling inhibitor can inhibit PD-1 and/or an activating ligand of PD-1, such as PD-L1 or PD-L2. The PD-1 signaling inhibitor can be a small molecule, a polynucleotide or a protein, such as an antibody. Preferably, the PD-1 signaling inhibitor is a monoclonal antibody, more preferably a humanized or fully human monoclonal antibody. In one embodiment, the PD-1 signaling inhibitor is an anti-PD-1 monoclonal antibody. In another embodiment, the PD-1 signaling inhibitor is an anti-PD-L1 monoclonal antibody. In another embodiment, the PD-1 signaling inhibitor is an anti-PD-L2 monoclonal antibody. Suitable PD-1 signaling inhibitors include, but are not limited to, those described in U.S. Pat. No. 8,008,449, WO 2006/121168, WO 2009/114335, U.S. Pat. Nos. 8,354,509, 8,609,089, US 2010/0028330, US 2012/0114649, WO 2007/005874, WO 2010/077634, U.S. Pat. No. 7,943,743, US 2012/0039906 and WO/2011/066342, each of which is incorporated by reference herein in its entirety. Examples of suitable PD-1 signaling inhibitors include YW243.55.570, MPDL3280A, MEDI-4736, MSB-0010718C, MDX-1105, and AMP-224. Preferred PD-1 signaling inhibitors include pembrolizumab (KEYTRUDA™), nivolumab (OPDIVO™), atelolizumab (TECENTRIQ™), avelumab (BAVENCIO™), durvalumab (MIFINZI™), ipilimumab (YERVOY®) and pidilizumab.

In particularly preferred embodiments of the invention, the PD-1 signaling inhibitor is pembrolizumab or nivolumab.

The cancer which is susceptible to immunotherapy is, for example, a cancer which is treatable with an immune checkpoint inhibitor. Representative cancers include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Glioblastoma, Childhood; Glioblastoma, Adult; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell Lung Cancer, Small Cell Lung Cancer; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Childhood, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macroglobulinemia; and Wilms' Tumor.

### In Vitro Production of A. Muciniphila

In a further embodiment, the present invention provides a method of producing A. muciniphila or enhancing the proliferation of A. muciniphila in vitro. The method comprises culturing A. muciniphila in the presence of an effective amount of a hydrogel as described herein. The A. muciniphila can be cultured, for example, in a suitable culture medium, such as an anaerobic broth, which comprises an effective amount of the hydrogel. The culture medium preferably comprises an amount of mucins effective to support the growth of the A. muciniphila, for example about 0.01% mucins or greater, preferably about 0.05% mucins or greater. In one embodiment, the concentration of mucins is from about 0.01% to about 0.1%, about 0.01% to about 0.07%, 0.03% to about 0.07% or about 0.03%. The culture medium can further comprise an amount of glucose sufficient to support the growth of A. muciniphila.

The A. muciniphila produced by the method of the invention can be used via methods known in the art to produce active A. muciniphila, live A. muciniphila, heat-inactivated A. muciniphila, pasteurized A. muciniphila, tindallized A. muciniphila, A. muciniphila cell lysate and fractions of the same, A. muciniphila cell-free supernatant, products of A. muciniphila fermentation, A. muciniphila fragments, A. muciniphila microbial metabolites, A. muciniphila-derived outer membrane vesicles (OMVs) and extracellular vesicles, and A. muciniphila exopolysaccharides.

The present invention can be further understood in view of the following non-limiting examples.

### EXAMPLES

### Example 1—Preparation of GelB

GelB was prepared according to the general method set forth in Example 1 of US 2016/0222134.

For the mixing step, a homogeneous mixture of sodium carboxymethylcellulose (AQUALON™ 7H4FM) (6% w/w DI Water), citric acid (0.2% w/w CMCNa), and DI water was obtained using a planetary mixer. Three (3) hours of mixing were enough to prevent any lumps in the mixture. For the drying step, a thin layer of CA/CMC/Water mixture was rolled out on a silicone sheet. The homogeneity of the layer is important to promote homogeneous drying and to prevent any residual stress in the material. The drying temperature was 70° C. For the first milling step, the dried material was ground by a cutting mill through 2 mm screen. For the first sieving, the ground material was sieved between 100-1600 microns. For the crosslinking step, 5 g of powder with a selected particle size of 100-1600 microns was placed in an aluminum dish and crosslinked at 120° C. for 4 hours. For the washing and drying step, the crosslinked powder was washed in DI water for 3 hours under constant stirring and then filtered and dried at 70° C. For the second milling step, the dried crosslinked material was ground by a cutting mill through 1 mm screen. For the second sieving step, the ground material was sieved for the final selected particle size of 100-1000 microns. The average elasticity (G′) of three samples of GelB when determined as set forth in Example 2 was 1827 Pa. The average media uptake ratio of the three samples in Simulated Gastric Fluid/water (1:8) as determined according to the method of Example 2 was 77.

### Example 2—Materials and Methods for Characterizing Polymer Hydrogels of the Invention Using Carboxymethylcellulose (CMC) as an Example

### (a) Preparation of Simulated Gastric Fluid/Water (1:8)

Reagents used for preparation of SGF/water (1:8) solution are purified water, sodium chloride, 1M hydrochloric acid and pepsin.
1. To a 1 L graduated cylinder pour about 880 mL of water.
2. Place the cylinder on a magnetic stirrer, add a magnetic bar and start stirring.
3. Begin monitoring the pH of the water with a pH meter.
4. Add a sufficient amount of 1M hydrochloric acid to bring the pH to 2.1±0.1.
5. Add 0.2 g NaCl and 0.32 g pepsin. Leave the solution to stir until complete dissolution.
6. Remove the magnetic bar and the electrode from the cylinder.
7. Add the amount of water required to bring the volume to 900 mL.

### (b) Determination of Viscosity of Carboxymethylcellulose Solutions

### Equipment and Materials:

Constant temperature water bath.
Glass Bottle, 500 ml with a cap, diameter of the neck at least 80 mm.
Brookfield Viscometer, model Myr VR3000 (EC0208) or equivalent equipped with: Spindle L4.
Thermal printer (PRP-058GI).
Mechanical overhead stirrer with anchor stainless steel stirrer.
Chain clamp to secure glassware.
Lab spatula.
Aluminum crucible.
Analytical balance, capable of weighing to the nearest 0.001 g.
Calibrated balance, capable of weighing, to the nearest 0.1 g.
Purified water.

### Preparation of Test Samples:

Prepare three CMC/water solutions as described below:
1. Measure the moisture content of CMC powder as described in [B] below.
2. Calculate the amount of water required using the equation:

$$water required [ g ] = 3 * ( 99 - {LOD}_{a v e r a g e} ) .$$

3. Weigh the needed amount of water for preparing the CMC solution into a beaker.
4. Pour roughly half of this water into the bottle, with the rest of the water remaining in the beaker.
5. Place and tie up the bottle under the stirrer motor with a chain clamp.
6. Insert the stirrer.
7. Mix the sample to assure uniformity.
8. Weigh 3.0±0.1 g of CMC powder.
9. Pour the powder in small amounts into the bottle while mixing at low speed (ca. 600 rpm).
10. Mix for 2 minutes and set the mixing speed to 1000 rpm.
11. Mix for no less than 10 minutes but no more than 30 minutes.
12. Add the remaining water.
13. Mix for additional 30 minutes.
14. If the CMC is not dissolved completely, continue stirring.
15. Once all the CMC is dissolved remove the anchor stainless steel stirrer and place the cap on the bottle.
16. Place the flask in the constant temperature bath, at 25.0° C.±0.1° C., for at least 30 minutes but no longer than one hour.
17. Shake the bottle vigorously for 10 seconds. The solution is ready to be tested.

### Viscosity Measurement:

1. Determine viscosity of each sample according to the instructions for the viscometer. Allow rotation of spindle for exactly 3 minutes.
2. Determine the average viscosity of the three solutions.

### Determination of Loss on Drying

The moisture content of a carboxymethylcellulose or crosslinked carboxymethylcellulose is determined according to USP <731>, Loss on Drying.

### Instruments/Equipment

Moisture Analyzer Radwag, Model WPS 50S

Lab Spatula

Aluminum crucible

Desiccator with silica gel.

### Procedure

1. Place the sample in the desiccator for at least 12 hours.
2. Place the aluminum crucible on the scale pan of the moisture analyzer and tare the balance.
3. Accurately weigh 1.000±0.005 g of a sample in the aluminum crucible. The initial weight of the sample is Wᵢ.
4. Set the Moisture Analyzer to heat the sample at 105° C. for 30 minutes under ambient pressure and moisture.
5. Turn on the Moisture Analyzer and run the LOD program (30 min at 105° C.).
6. Weigh the sample. The final weight of the sample is W_{f}.

The LOD value is determined according to the equation:

$$LOD = ( {W}_{i} - {W}_{f} ) / {W}_{i} \times 100 \% .$$

The Loss on Drying is determined in triplicate, and the reported LOD is the average of the three values.

### (c) Determination of Particle Size Range

### Equipment and Materials:

Sieve Shaker Retsch, Model AS 200 basic

Stainless Steel Sieves with mesh sizes 1000 m and 100 m

Aluminum weighing pan

Laboratory stainless steel spatula

Calibrated balance, capable of weighing to the nearest 0.1 g.

### Procedure:

1. Weigh the empty sieves and the aluminum pan to the nearest 0.1 g.
2. Weigh out 40.0±0.1 g of powder.
3. Stack the test sieves with sizes 1000 and 100 m with larger pore size on the top and the smaller at the bottom. Assemble the aluminum pan at the bottom of the nest.
4. Pour the sample into the 1000 m sieve, at the top of the stack.
5. Place this stack between the cover and the end pan of the shaker, so that the sample remains in the assembly.
6. Turn on the main switch of the shaker.
7. Set knob UV2 of the shaker for continuous operation.
8. Turn the knob MN2 of the shaker to the right to increase the vibration height until 50.
9. Shake this stack with the shaker for 5 minutes.
10. Disassemble the sieve and reweigh each sieve.
11. Determine the percentage weight of test specimen in each sieve as described in paragraph 8.
12. After measuring the weight of the full and empty test sieves, determine, by difference, the weight of the material inside each sieve.
13. Determine the weight of material in the collecting pan in a similar manner.
14. Use the weight of sample contained in each sieve and in the collecting pan to calculate the % distribution with the following equation:

$$Wx \% = Wx / W sample ⋆ 100 \%$$

where:
Wx %=sample weight in each sieve or in the collecting pan, in percentage where the index “x” is:
“>1000” for particle size bigger than 1000 m.
“100-1000” for particle size between 100 and 1000 m.
“<100” for particle size smaller than 100 m.
Wsample=initial weight of test specimen.

### (d) Determination of Tapped Density

### Equipment and Materials:

100 mL glass graduated cylinder

100 mL glass beaker

Lab spatula

Mechanical tapped density tester, Model JV 1000 by Copley Scientific

Calibrated balance capable of weighing to the nearest 0.1 g.

### Procedure:

1. Weigh out 40.0±0.1 grams of test sample. This value is designated M.
2. Introduce the sample into a dry 100 mL glass graduated cylinder.
3. Carefully level the powder without compacting and read the unsettled apparent volume, V0, to the nearest graduated unit.
4. Set the mechanical tapped density tester to tap the cylinder 500 times initially and measure the tapped volume, V500, to the nearest graduated unit.
5. Repeat the tapping 750 times and measure the tapped volume, V750, to the nearest graduated unit.
6. If the difference between the two volumes is less than 2%, V750 is the final tapped volume, Vf, otherwise repeat in increments of 1250 taps, as needed, until the difference between succeeding measurements is less than 2%.

### (e) Determination of Elastic Modulus (G′)

The elastic modulus (G′) is determined according to the protocol set forth below. The rheometer used is a Rheometer Discovery HR-1 (5332-0277 DHR-1) by TA Instruments or equivalent, equipped with a Peltier Plate; a Lower Flat plate Xhatch, 40 mm diameter; and an Upper Flat plate Xhatch, 40 mm diameter.

### Procedure

1. Put a magnetic stir bar in a 100 mL beaker.
2. Add 40.0±1.0 g of SGF/Water (1:8) solution prepared as described above to the beaker.
3. Place the beaker on the magnetic stirrer and stir gently at room temperature.
4. Accurately weigh 0.250±0.005 g of crosslinked polymer (e.g., carboxymethylcellulose) powder using a weighing paper (Wᵢₙ).
5. Add the powder to the beaker and stir gently for 30±2 min with the magnetic stirrer without generating vortices.
6. Remove the stir bar from the resulting suspension, place the funnel on a support and pour the suspension into the funnel, collecting any remaining material with a spatula.
7. Allow the material to drain for 10±1 min.
8. Collect the resulting material.
9. Subject the material to a sweep frequency test with the rheometer and determine the G′ value at an angular frequency of 10 rad/s.
   The determination is made in triplicate. The reported G′ value is the average of the three determinations.

### (f) Determination of Media Uptake Ratio (MUR) in SGF/Water (1:8)

The media uptake ratio of a crosslinked carboxymethylcellulose in SGF/water (1:8) is determined according to the following protocol.
1. Place a dried fritted glass funnel on a support and pour 40.0±1.0 g of purified water into the funnel.
2. Wait until no droplets are detected in the neck of the funnel (about 5 minutes) and dry the tip of the funnel with an absorbent paper.
3. Place the funnel into an empty and dry glass beaker (beaker #1), place them on a tared scale and record the weight of the empty apparatus (Wₜₐᵣₑ).
4. Put a magnetic stir bar in a 100 mL beaker (beaker #2); place beaker #2 on the scale and tare.
5. Add 40.0±1.0 g of SGF/Water (1:8) solution prepared as described above to beaker #2.
6. Place beaker #2 on the magnetic stirrer and stir gently at room temperature.
7. Accurately weigh 0.250±0.005 g of crosslinked carboxymethylcellulose powder using a weighing paper (Wᵢₙ).
8. Add the powder to beaker #2 and stir gently for 30±2 min with the magnetic stirrer without generating vortices.
9. Remove the stir bar from the resulting suspension, place the funnel on a support and pour the suspension into the funnel, collecting any remaining material with a spatula.
10. Allow the material to drain for 10±1 min.
11. Place the funnel containing the drained material inside beaker #1 and weigh it (W′_{fin}).

The Media Uptake Ratio (MUR) is calculated according to:

$$MUR = ( {W}_{fin} - {W}_{i n} ) / {W}_{i n} .$$

W_{fin} is the weight of the swollen polymer hydrogel calculated as follows:

$${W}_{fin} = {{W}^{′}}_{fin} - {W}_{tare} .$$

wherein Wᵢₙ is the weight of the initial dry sample. The MUR is determined in triplicate for each sample of crosslinked carboxymethylcellulose and the reported MUR is the average of the three determinations.

### Example 3—GelB Treatment Results in a Rapid and Sustained Increase in A. muciniphila when Preventatively Administered in a Mouse Model of High-Fat Diet Induced Obesity

Methods: C57B6/J mice were fed a low-fat diet (10% fat, 70% CHO) or high-fat diet (HFD; 45% fat, 36% CHO) for 18 weeks. In addition, two groups of animals were given HFD supplemented with either a low (2%) or high (4%) dose of GelB. Animal weight was monitored, and fecal samples were collected for microbiome analysis at baseline, months 1-3, and at sacrifice (week 18).

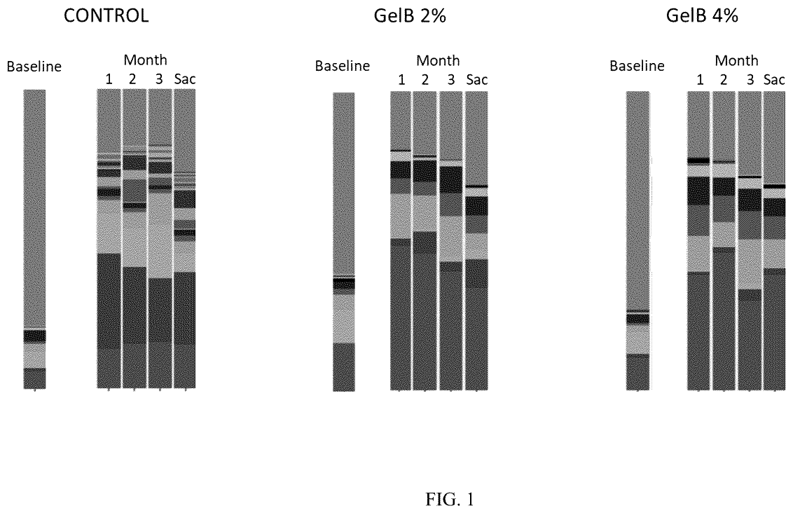

Results: GelB treatment resulted in increased relative abundance of A. muciniphila in both the 2% and 4% GelB groups compared to animals on control diet. Increase in A. muciniphila was rapid, and a relatively steady state of this species was achieved, as indicated by the lack of change through the duration of the experiment. The results are illustrated in FIG. 1, a stacked bar chart showing the relative abundance of A. muciniphila in mice treated with either low-fat control diet or high fat diet containing either 2 or 4% GelB. The relative abundance of A. muciniphila is represented by the bottom segment of each bar. GelB treatment at 2 and 4% resulted in increased A. muciniphila. This increase was rapid (evident at 1 month) and sustained until sacrifice after 18 weeks treatment.

### Example 4—GelB Treatment Results in Increased A. muciniphila in Both the Feces and Ileum Contents when Administered Therapeutically in a Mouse Model of High-Fat Diet Induced Obesity

Methods: C57BL/6J mice consumed a high fat diet (HFD; 45%) or standard chow for 12 weeks prior to treatment with either 2 or 4% GelB, AQUALON 7H4FM carboxymethylcellulose (CMC; 2%), HFD alone, or chow alone. Animals remained on treatment for an additional 12 weeks.

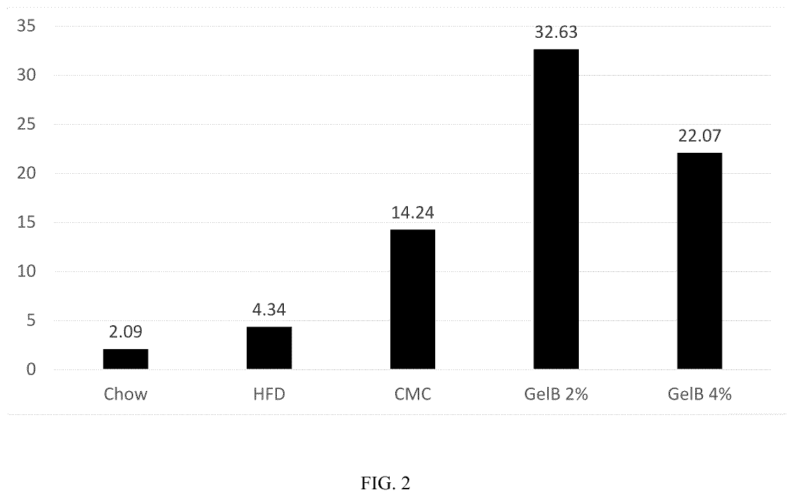

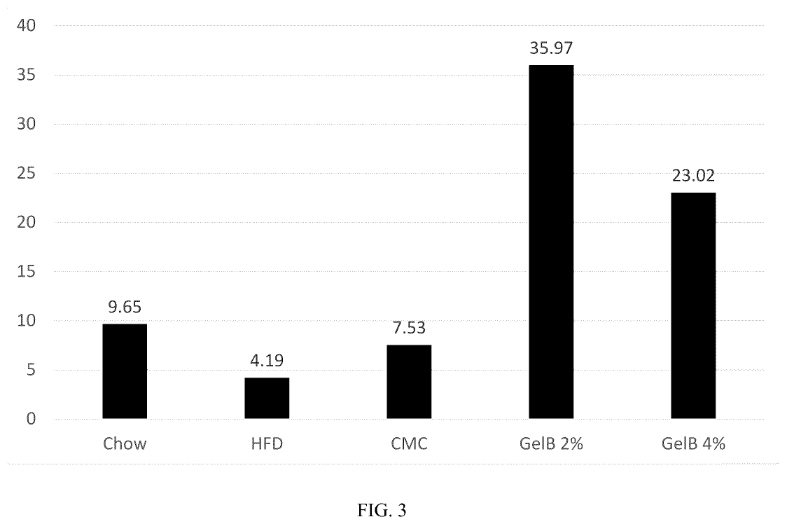

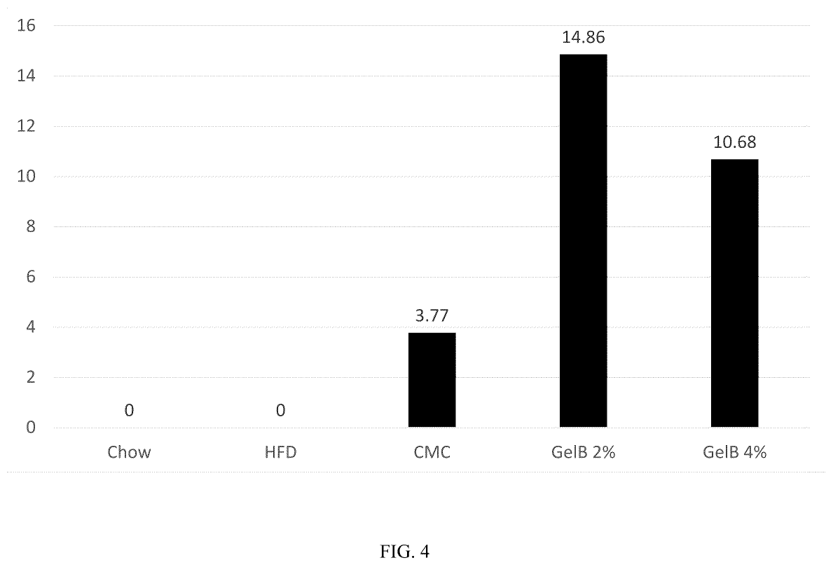

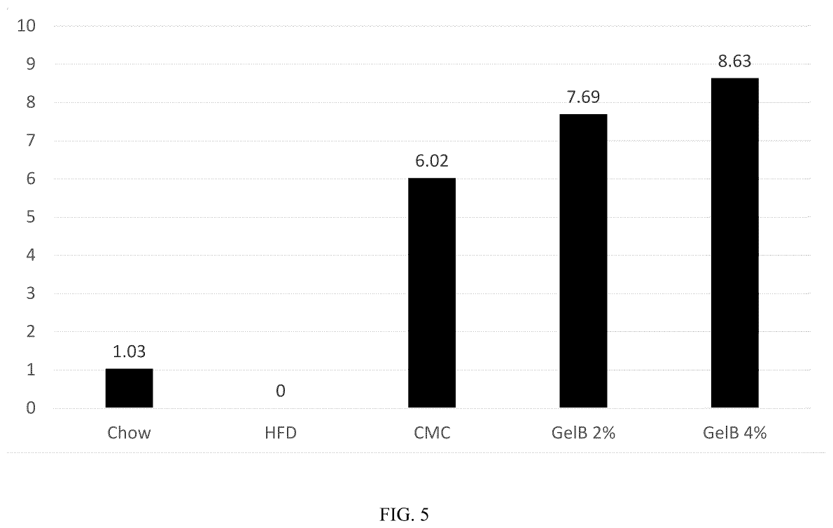

Results: After 4 weeks treatment, there was a profound shift in the abundance of A. muciniphila in the feces (FIGS. 2 and 3) and ileum contents (FIGS. 4 and 5) in animals receiving (a) the chow diet (chow), (b) HFD, (c) HFD supplemented with CMC, HFD supplemented with 2% GelB and HFD supplemented with 4% GelB. Greater relative abundance of A. muciniphila was seen in animals consuming GelB than CMC at all time points in both the feces and ileum contents. In FIGS. 2-5, the y-axis represents relative abundance of A. muciniphila and the relative abundance for each group is shown above the bar.

FIG. 2 is a bar chart showing the relative abundance of A. muciniphila in the feces of the mice after 4 weeks treatment.

FIG. 3 is a bar chart showing the relative abundance of A. muciniphila in the feces of mice after 12 weeks treatment.

FIG. 4 is bar chart showing the relative abundance of A. muciniphila in the ilea of mice after 4 weeks treatment.

FIG. 5 is a bar chart showing the relative abundance of A. muciniphila in the ilea of animals after 12 weeks treatment.

### Example 5—Effect of GelB on the Relative Abundance of A. muciniphila is Increased when Therapeutically Administered in Conjunction with Diet Reversal in a Diet-Induced Obesity Model of Non-Alcoholic Steatohepatitis (NASH)

Methods: Male DIO-NASH (C57BL/6JRj, n=112) were fed the Gubra Amylin NASH (GAN, 40% fat) diet for 36 weeks before treatment start. Animals with confirmed liver fibrosis (n=14 animals per group) received GAN diet containing 20%, 30% or 40% fat, respectively, with or without GelB for 8 weeks. Fecal samples were taken at baseline (treatment start) and 6 weeks.

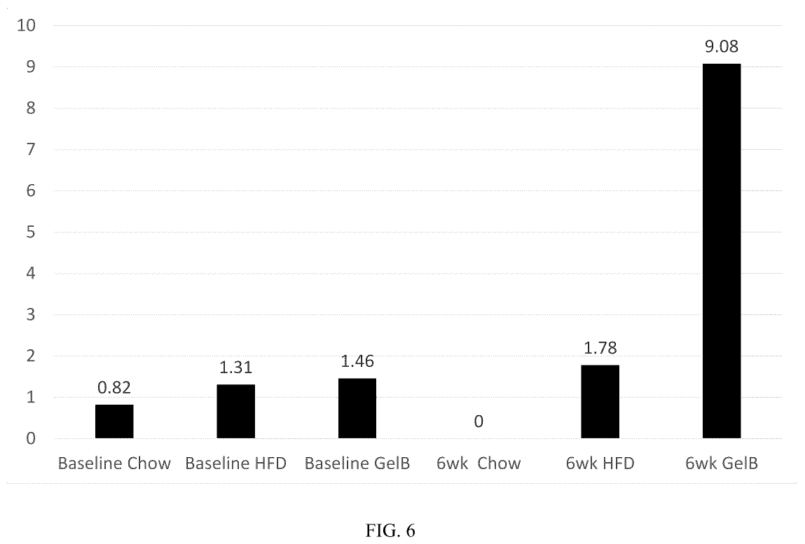

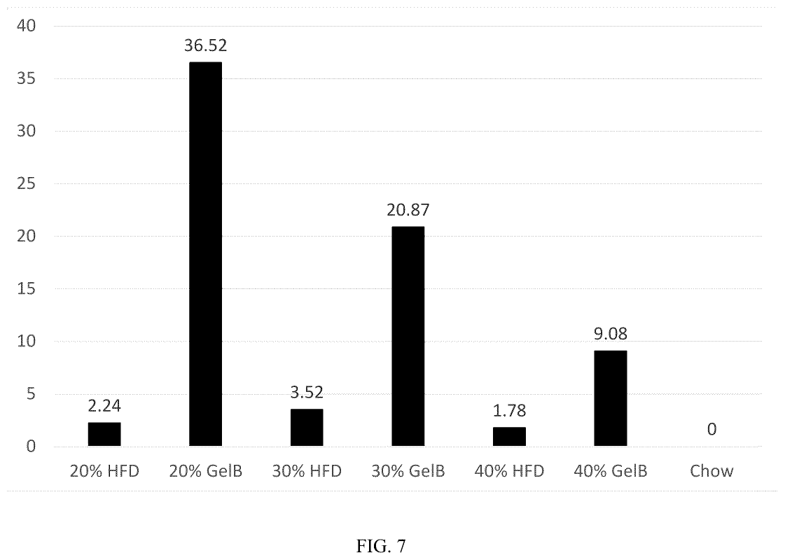

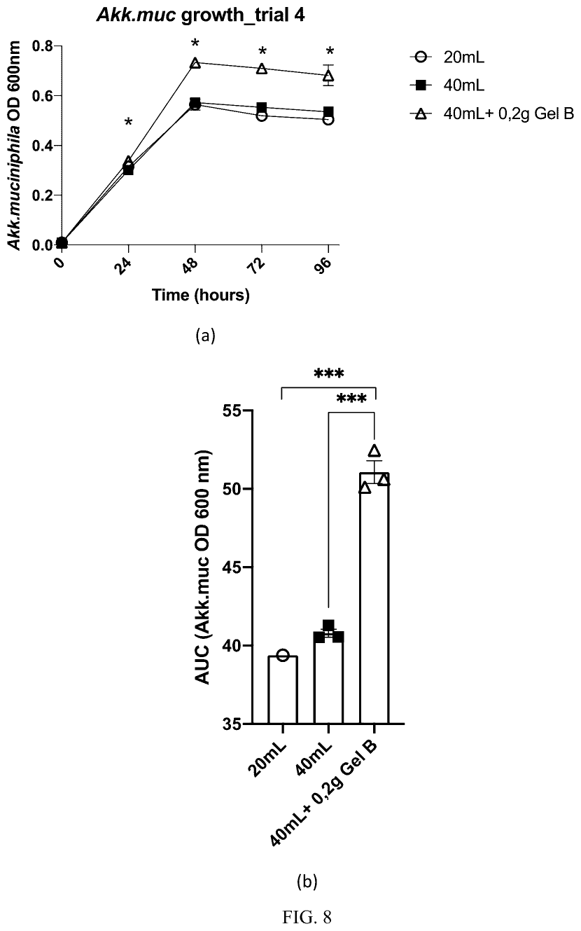

Results: After 6 weeks treatment, the relative abundance of A. muciniphila increased in animals consuming GelB compared to controls (FIG. 6). When GelB was administered in addition to a diet reversal, the abundance of A. muciniphila increased as dietary fat percentage decreased (FIG. 7). In FIGS. 6 and 7, the y-axis represents relative abundance of A. muciniphila and the relative abundance for each group is shown above the bar. The GAN diet is denoted in FIGS. 6 and 7 by HFD.

FIG. 6 is a bar chart showing the relative abundance of A. muciniphila in animals at baseline and after 6 weeks treatment in the setting of NASH.

FIG. 7 is a bar chart showing the relative abundance of A. muciniphila in animals receiving diet reversal after 6 weeks treatment in the setting of NASH.

### Example 6—Growth of A. muciniphila in the Presence of Hydrogels and Fibers In Vitro

The ability of certain hydrogels and fibers to support the growth of A. muciniphila in vitro was investigated. The protocol for this study is set forth below.

### Hydrogels and Fibers

Experiment 1: GelB, Inulin, Glucomannan, Guar gum. Psyllium
Experiment 2: GelB; Sodium carboxymethylcellulose—AQUALON 7H4FM; AVANTRX sodium carboxymethylcellulose; citric acid
Experiment 3: GelB with varying mucin concentrations

### Reagents and Equipment Required:

Sterile anaerobic broth (Thermo Scientific™ CM0957B) supplemented with 0.05% mucins and 4 g/L glucose.
50 mL conical sterile tubes.
Anaerobic hood/incubator.
Spectrophotometer.

### Technical Considerations Before Starting:

Akkermansia muciniphila is a strictly anaerobic bacteria (Gram−), with a slow duplication rate in vitro. It can precipitate forming black deposits at the base of culture tubes, therefore a gentle resuspension of bacteria is needed before OD₆₀₀ measurement.
Medium Uptake Ratio (MUR) must be assessed for each compound to assure a correct dosage (concentration in culture).
Maximum concentration of the compounds that can be tested is the one that occupies 50% of total liquid volume after reaching the equilibrium (i.e. 20 mL hydrated GelB and 20 mL liquid medium for a final 40 mL volume). Use the same dry weight to test matching compounds.

### Procedure:

1) Start A. muciniphila culture from stock. Measure the OD₆₀₀ until it reaches OD₆₀₀=0.5 (after about 72 hours).
2) Prepare sterile tubes with the different hydrogels and fibers (dry weight based on MUR) and fill them with 40 mL complete sterile anaerobic culture broth.
3) Subculture A. muciniphila culture at OD₆₀₀=0.01 in the tubes containing the different compounds to be tested.
4) Measure OD₆₀₀ every 24 hours till the culture reaches the plateau phase.

### Results

### Experiment 1

GelB induced the greatest increase in A. muciniphila, followed by medium and inulin, which induced similar increases in A. muciniphila, and psyllium. Guar gum and glucomannan both resulted in no increase of A. muciniphila.

### Experiment 2

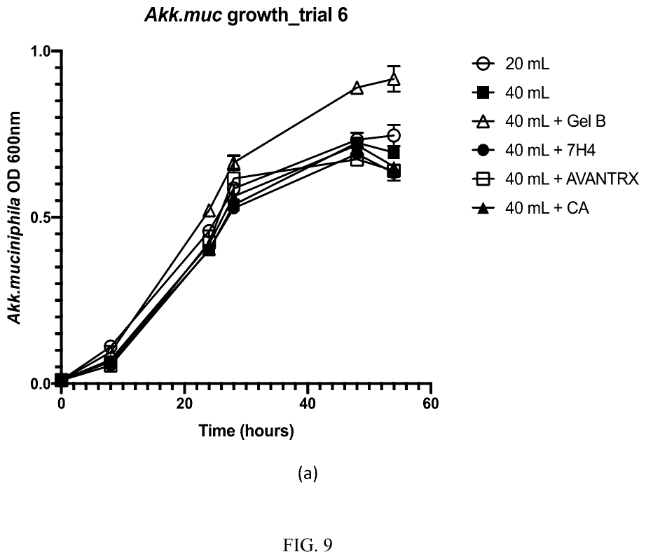

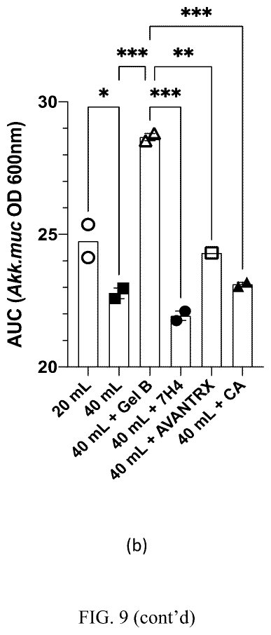

This study compared the proliferation of A. muciniphila in the presence of GelB, 7H4FM sodium carboxymethylcellulose (7H4), AVANTRX sodium carboxymethylcellulose (AVANTRX) and citric acid (CA). The results of this study are shown in FIG. 9. The presence of GelB resulted in the greatest increase in A. muciniphila, while the sodium carboxymethylcelluloses and citric acid showed results indistinguishable from medium alone (labelled 20 mL and 40 mL). This indicates that the three-dimensional structure of the hydrogel, not the individual components of the hydrogel, is important for inducing the growth of A. muciniphila.

### Experiment 3

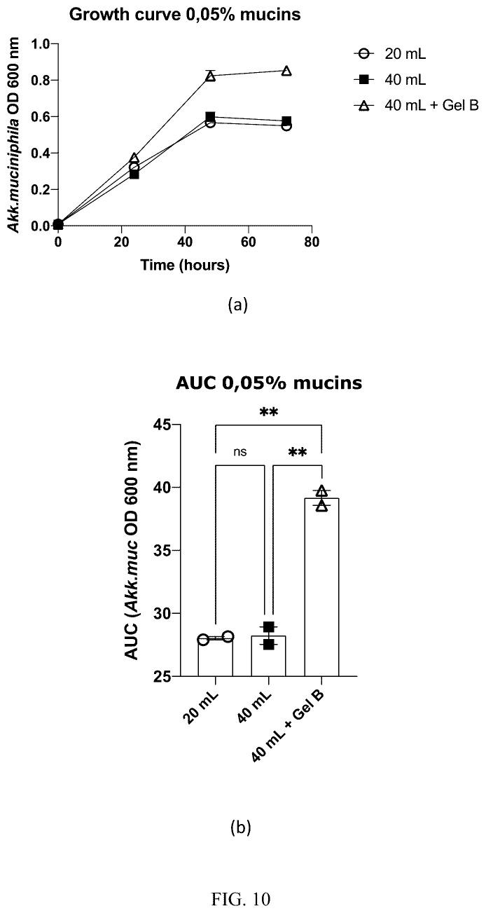

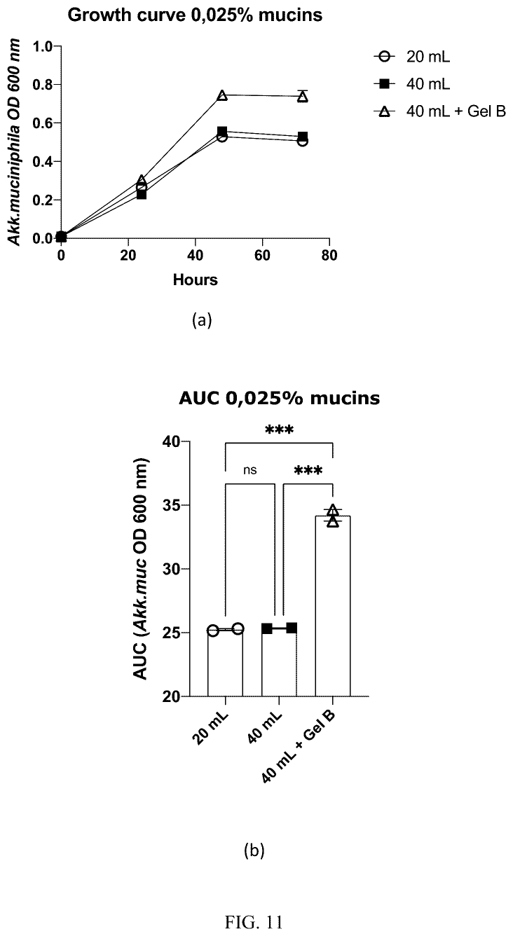

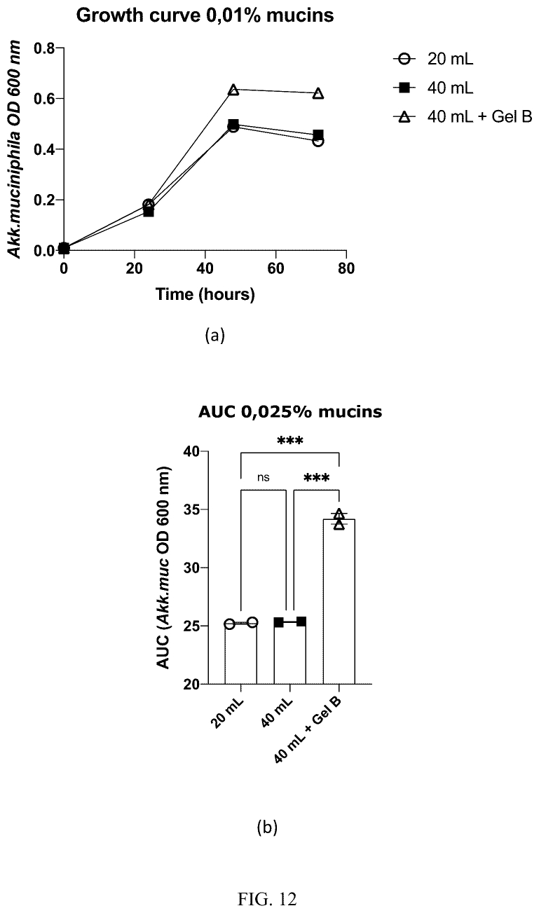

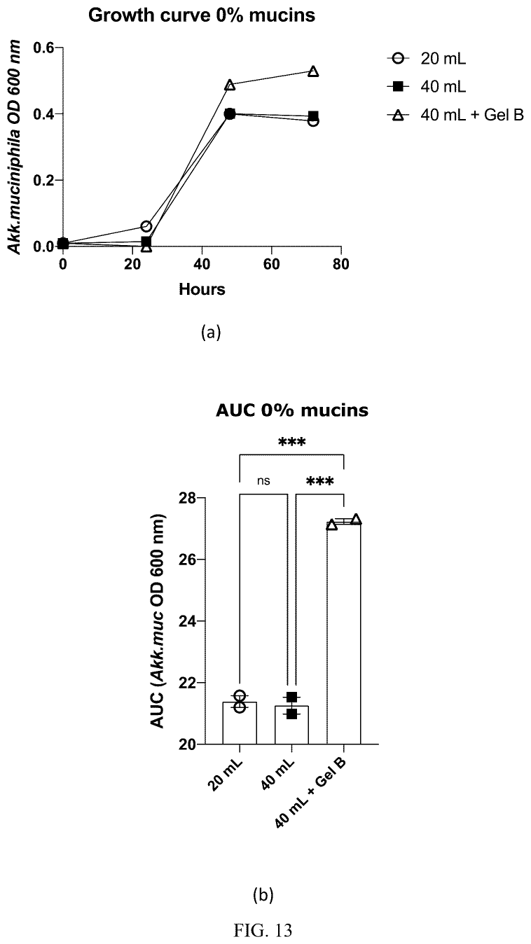

This study compared growth of A. muciniphila in the presence of GelB in sterile anaerobic broth supplemented with 0.05%, 0.025%, 0.01% or no mucins. The results are presented in FIGS. 10 to 13, which show that the presence of GelB increased A. muciniphila growth compared to media alone at all mucin levels.

### Example 7—Akkermansia muciniphila (AM) Supplemented GelB Results in Decreased Weight Gain Compared to Controls in a Treatment Model of High Fat Diet Induced Obesity

### Methods

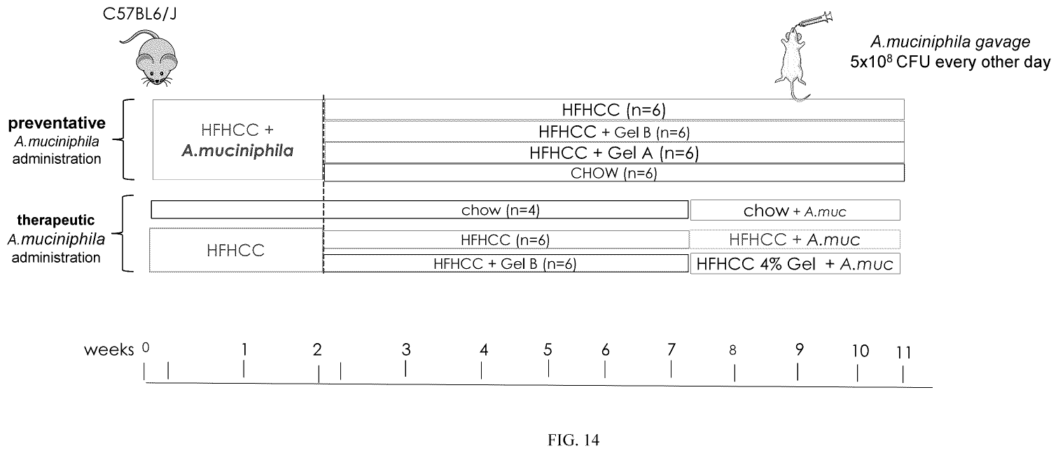

C57B6/J mice were fed chow or high-fat, high cholesterol diet (HFHCC; 39.6% fat, 1% cholesterol, 42 g/L fructose/glucose ad lib) during the experiment. One cohort of animals consuming either chow, high fat high cholesterol diet (HFHCC), or HFHCC plus 4% GelB received preventative AM (5×10⁸ CFU every other day) for 2 weeks at the beginning of the experiment. Another cohort of animals consumed either chow, HFHCC, or HFHCC+4% GelB, and received AM (5×10⁸ CFU every other day) starting at day 42 (FIG. 14). Animal weight was monitored weekly.

### Results

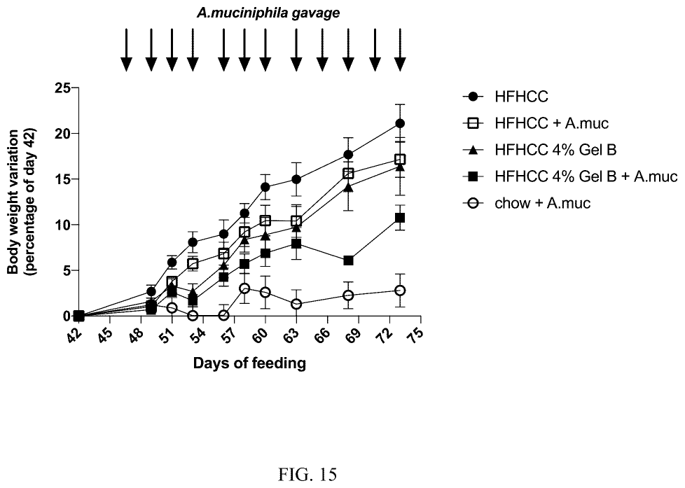

FIG. 15 compares the body weight variation during the therapeutic treatment of AM. Weight gain is plotted as percent weight gain from experimental day 42, when AM gavages were given to animals in the treatment cohort every other day. Animals consuming GelB alone or receiving AM alone gained slightly less (not significant) weight than HFHCC controls. Animals consuming GelB treated with AM, however, gained significantly less weight than HFHCC controls (p<0.0001), HFHCC plus GelB alone (p=0.0336), and HFHCC plusAM alone (p=0.0101).

### CONCLUSION

The addition of AM to GelB in animals consuming HFHCC led to decreased weight gain compared to animals consuming HFHCC plus GelB alone and HFHCC plus AM alone.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts, and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A method for increasing the level of A. muciniphila in the gut microbiome of a subject in need thereof, comprising administering to the intestinal tract of the subject (i) a polymer hydrogel having an elastic modulus (G′) of at least about 200 Pa and (ii) a probiotic, wherein the polymer hydrogel and the probiotic are administered in amounts which in combination are effective to increase the level of A. muciniphila in the gut microbiome of a subject.

2. A method for treating overweight, obesity, type 2 diabetes, non-alcoholic fatty liver disease or non-alcoholic steatohepatitis in a subject in need thereof, comprising administering to the intestinal tract of the subject therapeutically effective amounts of (i) a polymer hydrogel having an elastic modulus (G′) of at least about 200 Pa and (ii) a probiotic, wherein the polymer hydrogel and the probiotic are administered in amounts which in combination are therapeutically effective.

3. The method of claim 1, wherein the elastic modulus (G′) is about 200 Pa to about 8,000 Pa.

4. (canceled)

5. The method of claim 1, wherein the polymer hydrogel comprises a crosslinked polysaccharide.

6. The method of claim 5, wherein the polysaccharide is carboxymethylcellulose.

7. (canceled)

8. The method of claim 6, wherein the carboxymethylcellulose is crosslinked with a polycarboxylic acid or a bifunctional PEG.

9. (canceled)

10. The method of claim 6, wherein the carboxymethylcellulose has a viscosity as a 1% solution in water at 25° C. of at least 6000 cps.

11. The method of claim 10, wherein the carboxymethylcellulose is crosslinked with a polycarboxylic acid.

12. The method of claim 11, wherein the polycarboxylic acid is citric acid.

13. (canceled)

14. The method of claim 2, wherein the probiotic is selected from the group consisting of viable A. muciniphila, pasteurized A. muciniphila and a mixture of Lactobacillus rhamnosus LMG S-29148 and Bifidobacterium animalis subsp. lactis LMG P-281149.

15. (canceled)

16. The method of claim 2, wherein the polymer hydrogel and the probiotic are administered as separate compositions.

17. (canceled)

18. The method of claim 2, wherein the polymer hydrogel and the probiotic are administered in a single dosage form.

19. A composition comprising (i) a polymer hydrogel having an elastic modulus (G′) of at least about 200 Pa and (ii) a probiotic, wherein the polymer hydrogel and the probiotic are present in the composition in amounts which in combination are effective to increase the A. muciniphila level in the gut microbiome of a subject following administration of the composition to the intestinal tract of the subject.

20. The composition of claim 19, wherein the elastic modulus (G′) is about 200 Pa to about 8,000 Pa.

21. (canceled)

22. The composition of claim 19, wherein the polymer hydrogel comprises a crosslinked polysaccharide.

23. The composition of claim 22, wherein the polysaccharide is carboxymethylcellulose.

24. (canceled)

25. The composition of claim 23, wherein the carboxymethylcellulose is crosslinked with a polycarboxylic acid or a bifunctional PEG.

26. (canceled)

27. The composition of claim 23, wherein the carboxymethylcellulose has a viscosity as a 1% solution in water at 25° C. of at least 6000 cps.

28. (canceled)

29. (canceled)

30. The composition of claim 19, wherein the probiotic is selected from the group consisting of viable A. muciniphila, pasteurized A. muciniphila and a mixture of Lactobacillus rhamnosus LMG S-29148 and Bifidobacterium animalis subsp. lactis LMG P-281149.

31. (canceled)

32. (canceled)
